(19) United States
(12) Patent Application Publication
(10) Pub. No.: US 2010/0047790 A1
(43) Pub. Date: Feb. 25, 2010

(54) Sample analyser

(76) Inventors: Edwin Southern, Oxfordshire (GB); Natalie Milner, Oxfordshire (GB); Kaajal Patel, Oxfordshire (GB)

Correspondence Address: WENDEROTH, LIND & PONACK, L.L.P., 1030 15th Street, N.W.,, Suite 400 East, Washington, DC, 20005-1503, US

(21) Appl. No.: 12/448,345

(22) PCT Filed: Dec. 21, 2007
(86) PCT No.: PCT/GB2007/004961

§ 371 Date: Jun. 18, 2009

(30) Foreign Application Priority Data
Dec. 21, 2006 (GB) 0625995.4

(51) Int. Cl. C12Q 1/68; G01N 33/567; C12M 1/34

(52) U.S. Cl. 435/6; 435/7.2; 435/287.1; 435/287.2

(57) ABSTRACT

There are provided processes for analysing a plurality of different samples. The processes comprise the steps of: a) applying the samples to a support, to which an analytical component is immobilised; and b) allowing the samples to interact with the analytical component, thus permitting analysis of the samples. The samples are applied in step a) to different areas of the support to produce a spatial arrangement of samples on the support. The spatial arrangement of the samples is maintained in step b), thus permitting the results of the analysis to be matched to individual samples.

All documents cited herein are incorporated by reference in their entirety.

### TECHNICAL FIELD

This invention is in the field of sample analysis, in particular parallel analysis of biological samples.

### BACKGROUND ART

Parallel analysis of samples is important in many areas of technology, including biological research. Some known methods of parallel analysis involve analysing different samples separately in parallel, for example analysing different samples in different wells of a microtiter plate. Other known methods analyse the samples together, but require differential labelling of different samples so that the signal generated by each sample can be identified. DNA microarrays have been used for simultaneous parallel analysis of differentially labelled samples (for example, see reference 1).

There is a need for new and improved processes and devices for parallel analysis of samples, in particular biological samples. It is an object of the invention to provide such processes and devices.

### DISCLOSURE OF THE INVENTION

The invention provides processes and devices for parallel analysis of samples, in particular biological samples. In the methods of the invention, samples are analysed by allowing them to interact with an analytical component on a support. Target analytes in the samples are detected when the sample interacts with the analytical component.

The invention provides processes for analysing a plurality of different samples. The processes comprise the steps of: a) applying the samples to a support, to which an analytical component is immobilised; and b) allowing the samples to interact with the analytical component, thus permitting analysis of the samples. The samples are applied in step a) to different areas of the support to produce a spatial arrangement of samples on the support. The spatial arrangement of the samples is maintained in step b), thus permitting the results of the analysis to be matched to individual samples. This general approach is illustrated schematically in FIG. 1.

The methods of the invention involve the generation and maintenance of a spatial arrangement of samples on a support, which provides advantages over known methods for parallel sample analysis. For example, the methods of the invention permit multiple samples to be analysed in parallel using the same analytical component, such that each sample is subjected to substantially the same treatment and analysis, allowing direct comparison of results. In addition, the methods of the invention do not require differential labelling of different samples—the areas of the support where individual samples are located will be known or can be identified, so the signal generated by each individual sample can readily be identified.

In some embodiments, different analytical components are immobilised in different patches on the support. In those embodiments, multiple samples can be analysed in parallel for multiple target analytes using the same support, as illustrated schematically in FIG. 2.

The methods of the invention are useful for analysis of biological samples, such as samples containing cells or material derived from cells. The methods of the invention are particularly useful for analysis of individual cells or material derived from individual cells.

In one embodiment, the invention provides a process for analysing a plurality of different individual cells, comprising the steps of: a) applying material derived from individual cells to a support, to which an analytical component is immobilised; and b) allowing the material to interact with the analytical component, thus permitting analysis of the material. The material derived from different individual cells is applied in step a) to different areas of the support to produce a spatial arrangement of material on the support, and the spatial arrangement is maintained in step b), thus permitting the results of the analysis to be matched to individual cells.

In some methods, samples are applied directly to the support to generate a spatial arrangement of samples, as illustrated in FIG. 1. Thus, when the samples are biological samples comprising cells, the step a) of applying the samples to a support may comprise: (i) applying cells to the support; then (ii) releasing material from the cells. Alternatively, step a) may comprise: (i) releasing material from the cells; then (ii) applying the released material to the support. When cells are to be analysed individually, material derived from each cell will be applied to different areas of the support to produce a spatial arrangement of material on the support. The spatial arrangement of the material will be maintained in step b), so that the results of the analysis can be matched to individual cells. Such direct sample application methods are advantageous in some embodiments, because they can be performed using a simple device, and using a small number of sample handling steps.

In some methods, the samples are first applied to a transfer substrate to generate a spatial arrangement of samples, and then target analytes are transferred from the transfer substrate to the support. When a transfer substrate is used, the spatial arrangement of target analytes after transfer to the support matches the initial spatial arrangement of samples on the transfer substrate, thus permitting the results of the analysis to be matched to individual samples. This general approach is illustrated schematically in FIG. 3.

Such indirect sample application methods are advantageous in some embodiments, because it will not always be possible to easily generate and maintain a suitable spatial arrangement of samples on the support when the samples are applied directly to it. In addition, the transfer substrate may assist in sample preparation, by allowing transfer of target analytes to the support while preventing or reducing transfer of other components of the samples to the support.

Thus, the invention provides a process for analysing a plurality of different samples, comprising the steps of: a) applying the samples to different areas of a transfer substrate to produce a spatial arrangement of samples on the transfer substrate; then b) transferring target analytes from the transfer substrate to a support, to which an analytical component is immobilised; and c) allowing the target analytes to interact with the analytical component, thus permitting analysis of the samples. The spatial arrangement of the target analytes is maintained in steps b) and c), thus permitting the results of the analysis to be matched to individual samples.

Transferring target analytes from the transfer substrate to the support, while maintaining the spatial arrangement of target analytes, can be achieved in a variety of ways as described elsewhere herein. The transfer substrate can be positioned against or in close proximity to the support to facilitate transfer of target analytes from the substrate to the support. The transfer substrate and/or the support may be subjected to conditions which favour transfer of target analytes from the transfer substrate to the support. For example, an electrical potential or a magnetic field can be applied to the transfer substrate and/or the support, or reagents can be applied to the transfer substrate and/or the support, to facilitate transfer of target analytes from the substrate to the support.

The invention also provides devices and kits used in the methods of the invention.

The devices of the invention comprise a support, to which an analytical component is immobilised. During use, the devices of the invention comprise a support, to which an analytical component is immobilised, and on which support a plurality of samples are located in a spatial arrangement that permits the results of analysis using the analytical component to be matched to individual samples.

Thus, the invention provides a device for analysing a plurality of different individual cells, comprising a support, to which an analytical component is immobilised, and on which support material derived from a plurality of different individual cells is located in a spatial arrangement that permits the results of analysis using the analytical component to be matched to individual cells.

The invention also provides a device for analysing a plurality of different samples comprising: (i) a support, to which an analytical component is immobilised; and (ii) a transfer substrate positioned against or in close proximity to the support.

The invention also provides a kit for analysing a plurality of different samples, comprising: (i) a support, to which an analytical component is immobilised; and (ii) a material applicator, for applying a plurality of different samples to the support in a spatial arrangement that permits the results of analysis using the analytical component to be matched to individual samples.

The invention also provides a kit for analysing a plurality of different individual cells, comprising: (i) a support, to which an analytical component is immobilised; and (ii) a material applicator, for applying material derived from a plurality of different individual cells to the support in a spatial arrangement that permits the results of analysis using the analytical component to be matched to individual cells. The material applicator may be an applicator for applying individual cells to different areas of the support and then releasing material from the individual cells. Alternatively, the material applicator may be an applicator for releasing material from the individual cells and then applying the material released from individual cells to different areas of the support, e.g. a transfer substrate as described herein. As noted elsewhere herein, when cells are to be analysed individually, material derived from each cell will be applied to different areas of the support to produce a spatial arrangement of material on the support.

The invention also provides a kit for analysing a plurality of different samples, comprising: (i) a support, to which an analytical component is immobilised; (ii) a transfer substrate; and (iii) means for transferring target analytes from the transfer substrate to the support, which permits a spatial arrangement of samples on the transfer substrate to be maintained when target analytes are transferred to the support.

The dimensions and parameters of the various features of the devices and kits of the invention can vary according to particular needs and applications. Likewise, the precise steps of the methods of the invention can vary according to particular needs and applications. Different analyses can require different devices or processes within the scope of the invention. For instance, different sample types may require devices with different dimensions, or may require different sample preparation steps or different detection methods. Different analyses of the same sample type may use different analytical components e.g. for proteome analysis vs. transcriptome analysis. Moreover, devices can be designed and used based on previous experimental data. For example, if a device fails to give useful data in an initial experiment, variables such as the type of analytical component, temperature of operation, buffers, timings etc. can be altered in further experiments.

In some embodiments, the methods, devices and kits of the invention allow detection of individual target analyte molecules, such as individual mRNA molecules.

The processes and devices of the invention are described in more detail below.

### The Support

The devices of the invention comprise a support, to which an analytical component is immobilised.

The support may be constructed of any suitable material. The choice of materials for the support is influenced by a number of design considerations, and suitable materials can readily be selected by the skilled person based on the requirements of a particular device. For example, the material(s) should be stable to the reagents applied to the device during use, and compatible with the methods used for detecting the target analytes.

In some embodiments, materials impermeable to the reagents used during use of the device are used to construct the support (e.g., see Examples 1-10 and 13 herein).

In other embodiments, materials permeable to the reagents used during use of the device are used to construct the support (e.g., see Examples 11, 12 and 14 herein). Thus, the invention provides a device for analysing a plurality of different individual cells, comprising a support permeable to the reagents that are applied to the device during use, to which support an analytical component is immobilised, and on which support material derived from a plurality of different individual cells is located in a spatial arrangement that permits the results of analysis using the analytical component to be matched to individual cells. Such devices may comprise means for applying reagents to one or both faces of the support and/or means for removing reagents from one or both faces of the support. For example, the device may comprise one or more inlet(s) that permit reagents to be applied to one or both faces of the permeable support and/or one or more outlet(s) that permit reagents to be removed from one or both faces of the permeable support.

A permeable support may, for instance, be constructed from Nylon, nitrocellulose, GVHP, Immobilon-P or Immobilon-FL.

For some applications, it may be useful to attach components covalently to the support, so a suitable material should be selected by the skilled person. For some applications it will be desirable to use a hard material; other applications may need a flexible material. If fluorescence is to be used for detection, then the material should be transparent to the excitation and emission wavelengths, and also have low intrinsic fluorescence at these wavelengths. Materials that can propagate an illuminating evanescent wave (by total internal reflection) may be preferred for use with certain detection techniques.

Thus, supports of the invention can be made from a variety of materials, including but not limited to silicon oxides, polymers, ceramics, metals, etc. Specific materials that can be used include, but are not limited to: glass; polyethylene; PDMS; polypropylene; and silicon.

In the methods of the invention, samples are applied to a support, to which an analytical component is immobilised. Any support to which multiple samples can be applied to generate a spatial arrangement of samples, and to which an analytical component can be immobilised, can be used. The support will allow multiple samples to be analysed using a single patch of an analytical component. During use of the device, individual samples applied to a patch are in liquid communication, i.e. they interact with the same solution-phase reagents. The samples on a patch need not be in liquid communication throughout use of the device. The samples on a patch may be in liquid communication when the samples are applied to the support and/or when the samples are allowed to interact with the analytical component. The samples on a patch need not be in liquid communication at other stages of the methods of the invention, such as when the results of the analysis are recorded.

When different analytical components are immobilised in different patches on the same support, the arrangement shown in FIG. 1 is repeated, as desired.

In some embodiments, the support allows different patches on the support to be in liquid communication with each other during use of the device. For example, different patches may be arranged on a substantially planar surface, such as the surface of a glass microscope slide. Embodiments where different patches are in liquid communication with each other are advantageous in some embodiments, because they enable the same solution-phase reagents to be applied to the samples applied to different patches (e.g., for analysing different nucleic acid target analytes). The different patches need not be in liquid communication throughout use of the device, as described above.

In other embodiments, the support does not allow different patches to be in liquid communication with each other during use of the device, although the different samples applied to each individual patch are in liquid communication. For example, different patches may be arranged on a substantially non-planar surface, such as in the wells of a 96-well microtiter plate. During use, multiple samples could be applied to each well, generating a spatial arrangement of samples in each well. Embodiments where different patches are not in liquid communication with each other are advantageous in some embodiments, because they enable different solution-phase reagents to be applied to different analytical components, for analysing e.g. protein and nucleic acid target analytes on the same support.

In some embodiments, methods and devices in which different individual samples are not in liquid communication during use of the device, in particular during the sample application and/or analysis steps (e.g., the individual samples are applied to, or analysed in, different wells or channels of a support) are specifically excluded from the scope of this invention.

In some embodiments, methods and devices in which different patches are not in liquid communication during use of the device, in particular during the sample application and/or analysis steps (e.g., the patches are in different wells or channels of a support) are specifically excluded from the scope of this invention.

The maintenance of the spatial arrangement of samples in the methods of the invention allows the signal arising from each sample to be distinguished, even though the samples are applied to a single patch of an analytical component and are in liquid communication at some stage during use of the device. Supports suitable for use in the invention will be evident to the skilled person.

### The Analytical Component

The devices of the invention include an analytical component that can interact with target analytes in the samples to give analytical results. The devices may include single or different analytical components that can interact with different target analytes in the samples, such that the arrangement shown in FIG. 1 is repeated, as desired, at different areas of the device. A device comprising a single analytical component allows parallel analysis of multiple samples for a single type of target analyte using the same support. A device comprising different analytical components allows parallel analysis of multiple samples for multiple different target analytes using the same support.

The analytical components in any given device will generally be chosen based on knowledge of the sample type and target analytes of interest in order to give analytical data of interest. Typically, the analytical components will be biological molecules, such as nucleic acids for hybridisation, antibodies for antigen binding, antigens for antibody binding, lectins for binding to sugars and/or glycoproteins, etc. Analyses of genome, transcriptome, proteome, etc. can thus be performed.

Preferred analytical components are immobilised binding reagents, such as nucleic acids for hybridisation, antibodies for antigen binding, antigens for antibody binding, lectins for capturing sugars and/or glycoproteins, etc. Preferred analytical components are specific binding reagents, which are specific for a chosen target e.g. a nucleic acid sequence for specifically hybridising to a target of interest, an antibody for specifically binding a target antigen of interest. The degree of specificity can vary according to the needs of an individual experiment e.g. in some experiments it may be desirable to capture a target with nucleotide mismatch(es) relative to an immobilised sequence, but other experiments may require absolute stringency.

When the device comprises a single analytical component, the analytical component is preferably arranged in a discrete patch on the support, to facilitate data analysis.

When the device comprises a series of different analytical components, the different analytical components are preferably arranged in discrete patches on the support, to facilitate data analysis. If different analytical components are not separate then it may not be clear which of the different target analytes gives rise to an observed signal. It is possible, however, for neighbouring patches of different analytical components to overlap slightly, or not to have tight boundaries, provided that the signal arising from one patch can be distinguished from the signal arising from a different patch. In some embodiments, it may be advantageous for patches to overlap, or even for different analytical components to be immobilised on a single patch (see elsewhere herein).

When the device comprises a series of different analytical components, the different analytical components are preferably immobilised on a substantially planar surface (e.g. a glass microscope slide). However, devices having different analytical components immobilised in patches on different parts of a substantially non-planar surface (e.g. in different wells or channels) are also envisaged, as described elsewhere herein.

The device may include immobilised nucleic acids for capturing specific nucleic acids by hybridisation. The sequence of the nucleic acids will be chosen according to the target analyte(s) of interest. More preferably, the analytical components retain specific mRNA transcripts. The immobilised nucleic acids are preferably DNA, are preferably single-stranded, and are preferably oligonucleotides (e.g. shorter than about 500 nucleotides, <450 nt, <400 nt, <350 nt, <300 nt, <250 nt, <200 nt, <150 nt, <100 nt, <50 nt, or shorter).

The device may also include immobilised analytical components for capturing proteins. These will typically be immunochemical reagents, such as antibodies, although other specific binding reagents can also be used e.g. receptors for capturing protein ligands and vice versa. The use of aptamers for capturing proteins is envisaged.

It is also envisaged that the analytical component might be a small molecule, e.g. a small molecule drug candidate. Thus, the methods and devices of the invention can be used in small molecule screening assays, to identify a small molecule that interacts with a component of a sample (e.g. a small molecule that interacts with material derived from cells) or to identify a component of a sample that interacts with a small molecule. Preferably, the small molecule is an organic molecule with a molecular weight of less than 2000 Daltons, or less than 1500 Daltons, or less than 1000 Daltons, or less than 750 Daltons, or less than 500 Daltons, or less than 350 Daltons, or less than 250 Daltons. The small molecule may be a peptide or peptide analog, e.g. a peptide or peptide analog comprising at least 5 amino acid residues, at least 10 amino acid residues, at least 15 amino acid residues, at least 20 amino acid residues, at least 25 amino acid residues, or more. Thus, the methods and devices of the invention can be used in peptide and peptide analog screening assays, to identify a peptide or peptide analog that interacts with a component of a sample (e.g. a peptide or peptide analog that interacts with material derived from cells) or to identify a component of a sample that interacts with a peptide or peptide analog.

A single device of the invention can include analytical components for analysing both nucleic acids and proteins.

Methods for immobilising analytical components onto supports are well known in the art. Methods for attaching nucleic acids to supports in a hybridisable format are known from the microarray field e.g. attachment via linkers, to a matrix on the support, to a gel on the support, etc. The best-known method is the photolithographic masking method used by Affymetrix for in situ synthesis of nucleotides on a glass support, but electrochemical in situ synthesis methods are also known, as are inkjet deposition methods. Methods for attaching proteins (particularly antibodies) to supports are similarly known.

Immobilised nucleic acids can be pre-synthesised and then attached to a support, or can be synthesised in situ on a support by delivering precursors to a growing nucleic acid chain. Either of these methods can be used to construct a device of the invention. Preferred immobilised nucleic acids are formed by in situ synthesis using electrochemical deprotection of a growing nucleic acid chain (as described in references 2, 3 & 4).

One analytical procedure that can be used with the invention involves capture of mRNA by hybridisation to an immobilised capture DNA, followed by reverse transcription of the mRNA using the immobilised DNA as a primer. In this procedure, therefore, a reverse transcriptase has to be present, and this can be introduced together with dNTPs and other reagents after mRNA has been immobilised. The reverse transcription process extends the immobilised primer to synthesise an immobilised cDNA and thus leads to covalent modification of the device of the invention. In order to facilitate chain extension of a DNA on the device by reverse transcription, it will be immobilised via its 5′ end or via an internal nucleotide, such that it has a free 3′ end. Further details of this technique are given below.

The devices may contain one or more analytical component(s). For example, the devices may contain N different analytical components, wherein N is selected from 2, 3, 4, 5, 6, 7, 8, 9, 10, 15, 20, 25, 50, 75, 100, 125, 150, 175, 200, 250, 300, 400, 500 or more. The devices may contain at least 10^{N} different analytical components, wherein N is selected from 0, 1, 2, 3, 4, 5 or more. Immobilisation of at least 10⁶ different oligonucleotides onto a single support is well known in the field of microarrays. The N or 10^{N} different analytical components will typically be arranged in N or 10^{N} different patches on the support, respectively.

The devices may contain two or more patches of a single analytical component, such as 3 or more, 4 or more, 5 or more, 6 or more, 7 or more, 8 or more, 9 or more, or 10 or more patches of the same analytical component.

A patch of analytical component is sized to permit parallel analysis of at least two samples. Preferably, a patch is sized to allow 5 or more (such as 10 or more, 15 or more, 20 or more, 25 or more, 50 or more, 75 or more, 100 or more, 150 or more, or 200 or more) different samples to be applied to the patch, with adequate spacing to allow the signal arising from each sample to be distinguished.

Thus, in use the devices of the invention may comprise a support, to which an analytical component is immobilised, and on which support 5 or more (such as 10 or more, 15 or more, 20 or more, 25 or more, 50 or more, 75 or more, 100 or more, 150 or more, or 200 or more) different samples are located on a single patch of analytical component.

The patch size required to permit parallel analysis of samples will vary, depending on factors such as the volume of each sample, the spreading of the sample when applied to the support, the sensitivity and resolution of the detection equipment, and the number of samples to be analysed in parallel on a patch. Preferably, the patch size will allow multiple samples to be applied to distinct regions of the patch without overlapping (as in FIG. 1), to facilitate data analysis—if different samples are not adequately spaced then it will not be clear which of the samples gives rise to an observed signal. It is possible, however, for neighbouring samples to overlap slightly when applied to a patch, provided that the signal arising from one sample can be distinguished from that arising from another sample.

The average centre-to-centre separation of samples after application to a patch is preferably at least 2p, where p is the average longest dimension (length or diameter) of samples after application to a patch. For example, if samples have an average diameter of approximately 25 μm after application to a patch, the centre-to-centre separation of the samples will preferably be at least 50 μm. The centre-to-centre separation of samples after application to a patch may be 3p, 4p, 5p, 6p, 8p, 10p, or more.

The average centre-to-centre separation of samples after application to a patch is preferably at least 10^{Y} m, where Y is selected from −3, −4, −5, etc.

The desired centre-to-centre separation of samples on a patch may be achieved by appropriate dilution of a solution or suspension of different samples, as mentioned elsewhere herein. When analysing individual cells, it may be necessary to treat the cells to reduce cell clumping, to ensure the desired centre-to-centre separation.

The patch sizes in current microarrays range from about 1 μm diameter to about 1 mm diameter. In the devices of the invention, a patch preferably has an area of at least 10^{X} m², where X is selected from −2, −3, −4, −5, −6, −7, −8, −9, −10, −11, −12, etc. Microarrays with patch sizes in the order of 10 μm×10 μm (i.e. 10⁻¹⁰ m²) are readily prepared using current technology.

When the invention is used for parallel analysis of individual cells on a patch, a patch will be sized to permit at least two cells, or material derived from at least two cells, to be applied to a patch. This will generally require a patch with an area of >2a, where a is the mean cross-sectional area of the cell type(s) of interest. Usually, a patch will be >3a, >4a, >5a, >10a, >15a, >20a or >25a, to take into account the volume of each cell, the spreading of material derived from each cell, the sensitivity and resolution of the detection equipment, and the number of cells to be analysed in parallel on each patch (see Example 6 herein).

Typical cell and organelle dimensions are given in the following table:

| | | |
|---|---|---|
| S. cerevisiae | 5 | μm |
| S. pombe | 2 × 7 | μm |
| Mammalian cell | 10-20 | μm |
| Human T lymphocyte | 6-8 | μm |
| E. coli | 1 × 3 | μm |
| Mammalian mitochondrion | 1 | μm |
| Mammalian nucleus | 5-10 | μm |
| Plant chloroplast | 1 × 4 | μm |

Thus, depending on the cells to be analysed, a patch may have a longest dimension (length or diameter) of greater than 1 μm, such as greater than 3 μm, greater than 5 μm, greater than 10 μm, greater than 25 μm, greater than 50 μm, greater than 100 μm, greater than 250 μm, greater than 500 μm, greater than 750 μm, or greater than 1000 μm (1 mm).

For example, if the skilled person wishes to analyse 16 individual human T lymphocytes on a patch of analytical component, a square patch of >32 μm×32 μm (1024 μm²) will usually be required to ensure that the 16 cells can readily be individually analysed in parallel on the patch.

The skilled person can readily select appropriate patch sizes for the number and type of samples of interest. Larger patches will generally permit a larger number of individual samples to be analysed in parallel on the patch. Larger patches may also permit larger samples to be applied, while maintaining adequate sample spacing on the patch. Larger patches may also permit equivalent samples to be more easily resolved by the detection equipment, by allowing samples to be spaced further apart. However, larger patches may require a larger support, unless the total number of patches is reduced.

Patches within devices of the invention may have the same size, or different sizes.

The edge-to-edge separation of patches is preferably at least 10^{Y} m, where Y is selected from −3, −4, −5, etc. Adjacent patches may abut or may overlap, but it is preferred that adjacent patches are separated by a gap.

A patch preferably has a rectangular or square shape, but may also have a circular shape. In some embodiments, the shape and size of the patches will be determined by the characteristics of the support (e.g. when the support is a microtiter plate, the size and shape of the patches may match the size and shape of the well bases). Patches within devices of the invention may have the same shape, or different shapes.

The methods of the invention are particularly advantageous when used for parallel analysis of multiple samples per patch of analytical component. However, the invention can also be used for analysis of a single sample per patch of analytical component. For example, a single cell might be analysed on each patch of a device of the invention. Such an arrangement might be useful, for example, if the pattern of gene expression in a population of identical, synchronous cells is to be analysed. In that case, a single cell of the population can be analysed on each patch for a different target analyte. Thus, the invention also provides a process for analysing a plurality of individual cells, comprising the steps of: a) applying material derived from individual cells to a support, to which an analytical component is immobilised; and b) allowing the material to interact with the analytical component, thus permitting analysis of the material. The material derived from different individual cells is applied in step a) to patches of different analytical components on the support to produce a spatial arrangement of material on the support. The spatial arrangement is maintained in step b), thus permitting the results of the analysis to be matched to individual cells.

When a single cell is analysed on a patch of analytical component, the method of the invention has similarities to analysis by fluorescence in situ hybridisation (FISH). However, in the methods of the invention, a single cell is analysed on a support, to which an analytical component is immobilised. In contrast, in FISH, a single cell is analysed, but the analytical component is provided in the solution phase, such that different target analytes cannot readily be detected in parallel.

It is envisaged that the devices and methods of the invention might be used to select a sub-population of cells from a population of cells applied to the device. For example, individual cells at a particular stage of the cell cycle (i.e. synchronous cells) might be selected on the basis of cell-surface antigen expression using immobilised antibodies or aptamers. In such embodiments, each patch may comprise more than one analytical component (e.g. 2 or 3 different analytical components) to permit selection of multiple cells types, or selection of those cells which have certain combinations of cell-surface antigens, on a single patch. Selection of cells may require washing of the device to remove cell types that do not bind to the immobilised analytical components.

After selection of a subpopulation of cells, that subpopulation may be further analysed using the methods of the invention. For example, if a subpopulation of cells is selected on the basis of cell-surface antigen expression, that subpopulation of cells may then be lysed and the contents of the individual cells analysed in parallel as described herein. In such embodiments, each patch may comprise more than one type of analytical component (e.g. 2 or 3 types of analytical component) to permit selection and analysis of cells on a single patch, e.g. on a single patch having immobilised antibodies and nucleic acids.

In addition to the immobilised analytical components described above, it is envisaged that solution phase probes may be used with the devices and methods of the invention. Typically, solution phase probes will be applied to the device after capture of target analytes by the immobilised analytical component(s). Solution phase probes will generally be chosen based on knowledge of the sample type and target analytes of interest in order to give analytical data of interest. Typically, the probes will be biological molecules, as described elsewhere herein.

In some embodiments, the use of solution phase probes is advantageous, because it permits more detailed sample analysis. For example, after capture of mRNA using a patch of immobilised oligo dT, and generation of immobilised cDNA representing the whole of the polyA+ population of the cells (see the Examples herein), solution phase gene specific probes might be applied to the patch to permit identification and quantitation of specific mRNAs. As a further example, after capture of antigens using a patch of a non-specific analytical component (e.g. a relatively unspecific antibody), the captured antigens might be analysed in more detail using a solution phase probe (e.g. an antibody that binds specifically to an antigen).

In embodiments where solution phase probes are used, a set of multiple different solution phase probes may be used to analyse multiple different target analytes in parallel. These different solution phase probes may each be specific for an individual target analyte (e.g. specific for an individual gene) or may be specific for multiple related target analytes (e.g. specific for sequences conserved across genes), depending on the analysis required. A set of different solution phase probes may consist of at least 2, at least 5, at least 10, at least 25, at least 50, at least 100, at least 150, at least 200, at least 300, at least 400, at least 500, or at least 600 different probes.

When the target analyte is a nucleic acid (e.g. mRNA), the solution phase probes need not be gene specific and may e.g. identify nucleotide sequences shared by multiple different genes, or sequences shared by multiple different organisms.

When the target analyte is a nucleic acid, the solution phase probes may form primers for extension by a polymerase using the immobilised cDNA as a template. The primer sequences can be selected for gene specific or non-specific extension.

Different solution phase probes might be applied to different areas of a single patch (e.g. a patch with immobilised cDNAs), for example using a probe applicator that comprises channels or pins. A suitable probe applicator is described in U.S. design Pat. D 413,390. When more than one probe is used, a suitable labelling and detection method can be selected from those known in the art. For example, different probes labelled with different fluorescent dyes may be applied to a patch simultaneously. Alternatively, or in addition, the different probes may be applied serially. In this case, a first probe (or set of probes) is applied to the device, the signal generated observed, and the probe(s) removed. A second probe (or set of probes) is then applied to the device, the signal generated observed, and the probe(s) removed. These steps can be repeated as necessary with further probes.

### Other Features of the Device

The devices of the invention may also include:
One or more electrodes. Electrodes can be used to generate an electrical potential across a device, to cause electroporation of cells, sample transfer etc.
A piezoelectric device in order to lyse cells.
A light source, e.g. a laser. A laser can be used to lyse cells and/or for data collection.
A detector, e.g. a mass spectrometer.

### The Target Analyte

The methods of the invention can be used for identification and quantitation of various target analytes. The target analyte can be any chemical entity that the skilled person might wish to detect or quantitate in a sample. The methods of the invention can be used to analyse biological or non-biological target analytes. Preferably, the target analyte is a biological target analyte.

The invention is particularly suitable for analysis of biological target analytes for which microarray analysis has previously been described e.g. nucleic acids and polypeptides. Suitable nucleic acid target analytes include, but are not limited to, genomic DNA, plasmid DNA, amplification products (e.g. from PCR), cDNA and mRNA.

The methods of the invention involve analysis of samples for the presence or amount of target analytes. It will be understood that not all samples tested using the methods of the invention will contain target analytes. Thus, references herein to transferring target analytes, detecting target analytes etc. are not limited to situations in which the sample contains target analytes (e.g. an assay for a pathogen may produce a negative result, or negative controls may be analysed).

### The Samples

The methods of the invention can be used to analyse various types of sample.

The sample can be anything that the skilled person might wish to analyse for the presence or amount of target analytes. The methods of the invention can be used to analyse biological or non-biological samples. Preferably, the sample is a biological sample, such as a sample containing cells or material derived from cells.

Biological samples can comprise, or be derived from, a variety of organisms and cell types, including both eukaryotes and prokaryotes. For example, the invention can be used to analyse bacteria, or samples derived from bacteria, including, but not limited to: E. coli; B. subtilis; N. meningitidis; N. gonorrhoeae; S. pneumoniae; S. mutans; S. agalactiae; S. pyogenes; P. aeruginosa; H. pylori; M. catarrhalis; H. influenzae; B. pertussis; C. diphtheriae; C. tetani; etc. Within the eukaryotes, the invention can be used to analyse animal cells, plant cells, fungi cells (particularly yeasts), etc. and samples derived from such cells. Preferred animal cells of interest are mammalian cells. Preferred mammals are primates, including humans.

Specific cell types of interest, particularly for human cells, include but are not limited to: blood cells, such as lymphocytes, natural killer cells, leukocytes, neutrophils, monocytes platelets, etc.; tumour cells, such as carcinomas, lymphomas, leukemic cells; gametes, including ova and spermatozoa; heart cells; kidney cells; pancreas cells; liver cells; brain cells; skin cells; stem cells, including adult stem cells and embryonic stem cells; etc. Cell lines can also be analysed.

When the sample comprises cells or material derived from cells, each sample may comprise multiple cells or material derived from multiple cells, such that the invention is used for parallel analysis of different cell populations. Alternatively, each sample may comprise an individual cell or material derived from an individual cell, such that the invention is used for parallel analysis of individual cells.

Accordingly, in some embodiments each sample may comprise: less than 1×10⁸ cells, less than 1×10⁷ cells, less than 1×10⁶ cells, less than 1×10⁵ cells, less than 1×10⁴ cells, less than 1×10³ cells, less than 100 cells, less than 50 cells, less than 25 cells, less than 20 cells, less than 15 cells, less than 10 cells, less than 5 cells, less than 3 cells, or a single cell.

In other embodiments, each sample may comprise: more than 3 cells, more than 5 cells, more than 10 cells, more than 15 cells, more than 20 cells, more than 25 cells, more than 50 cells, more than 100 cells, more than 1×10³ cells, more than 1×10⁴ cells, more than 1×10⁵ cells, more than 1×10⁶ cells, more than 1×10⁷ cells, or more than 1×10⁸ cells.

In other embodiments, each sample may comprise: material derived from less than 1×10⁸ cells, material derived from less than 1×10⁷ cells, material derived from less than 1×10⁶ cells, material derived from less than 1×10⁵ cells, material derived from less than 1×10⁴ cells, material derived from less than 1×10³ cells, material derived from less than 100 cells, material derived from less than 50 cells, material derived from less than 25 cells, material derived from less than 20 cells, material derived from less than 15 cells, material derived from less than 10 cells, material derived from less than 5 cells, or material derived from less than 3 cells.

In other embodiments, each sample may comprise: material derived from more than 3 cells, material derived from more than 5 cells, material derived from more than 10 cells, material derived from more than 15 cells, material derived from more than 20 cells, material derived from more than 25 cells, material derived from more than 50 cells, material derived from more than 100 cells, material derived from more than 1×10³ cells, material derived from more than 1×10⁴ cells, material derived from more than 1×10⁵ cells, material derived from more than 1×10⁶ cells, material derived from more than 1×10⁷ cells, or material derived from more than 1×10⁸ cells.

The present invention is particularly suitable for the analysis of different individual cells, including both eukaryotic cells and prokaryotic cells. For example, when each sample comprises an individual cell or material derived from an individual cell, the invention can be used to analyse a plurality of cells which, although of the same type (e.g. a cell line), are asynchronous i.e. at different stages of the cell cycle. The invention can also be used to analyse a plurality of cells which are of the same type and are synchronous i.e. at the same stage of the cell cycle.

The devices of the invention can be used to analyse a single type of cell. The devices of the invention can also be used to analyse more than one, such as two or more, three or more, four or more, five or more, etc. types of cell. For example, the devices of the invention can be used to analyse samples containing different types of bacteria (e.g. food samples), or samples containing different types of human cells (e.g. blood or tissue samples).

It may be desirable for the methods of the invention to include a sample preparation step that permits separation of the cell type(s) of interest from other components of the samples. In particular, it may be desirable to separate the cell type(s) of interest from other cell types in the samples. For example, when a blood sample is to be analysed using the devices of the invention, it may be desirable to remove red blood cells from the sample prior to analysis of the white blood cells. Thus, in some embodiments, the methods of the invention may comprise a step of separating one or more cell type(s) of interest from other components in the starting sample(s). In particular, the methods of the invention may comprise a step of separating one or more cell type(s) of interest from other cell types in the starting sample(s). In some embodiments, separation of different cell types may be achieved using a transfer substrate as described elsewhere herein. Other suitable separation methods will be known to those of skill in the art (e.g. FACS). After separation of cells of interest from other components in the starting samples, the cells of interest can be analysed using the devices of the invention. The other components of the samples (i.e. those components from which the cells of interest were separated) may be discarded, or may themselves be analysed using the devices of the invention.

Preferably, after separation of one or more cell type(s) of interest from one or more other components of the samples, at least 75% or more (such as 80% or more, 85% or more, 90% or more, 95% or more, 97% or more, 98% or more, or 99% or more) of the resulting population of separated cells are cells of the desired type(s).

As well as analysing cellular contents, it is possible to analyse organelles in eukaryotic cells, and particularly nuclei (e.g. for transcription factors), mitochondria and plastids (e.g. chloroplasts). Organelles can be prepared from cells, and then analysed as described herein for whole cells.

### Applying Samples to a Support

In some methods of the invention, samples are applied directly to a support to generate a spatial arrangement of samples. Samples can be applied directly to a support by any suitable method, including but not limited to pipetting, printing, spotting and spreading. For example, samples can be applied to a support using a sample applicator of the type described in U.S. design Pat. D 413,390.

When the samples comprise cells, the cells can be applied to the support, then material released from the cells. Alternatively, material can be released from the cells and then the released material applied to the support.

Material can be released from cells by any suitable method. Both mechanical and chemical methods are envisaged; exemplary methods are described below.

For instance, a lysis solution can be applied to cells on the support, and the cells lysed in situ. Typical lysis solutions that can be used may comprise components such as: a surfactant e.g. an ionic detergent such as SDS when analysing nucleic acids, or a non-ionic detergent such as Triton-X100 when analysing proteins; an enzyme to digest proteins e.g. proteinase K; an enzyme to digest nucleic acids e.g. a DNase and/or RNase; an enzyme to digest saccharides (e.g. β(1-6) and β(1-3) glycanases, mannase); a chaotrope to inactivate enzymes and solubilise cellular components e.g. a guanidine salt, such as guanidinium isothiocyanate; an organic solvent (e.g. toluene, ether, phenylethyl alcohol DMSO, benzene, methanol, or chloroform); an antibiotic; a thionin; a chelating agent (e.g. EDTA); a basic protein (e.g. protamine, or chitosan) etc. Such reagents are commonly used in existing techniques for bulk cell lysis. The choice of reagent(s) will depend on the nature of the analytes of interest e.g. if the aim is to analyse mRNA then proteases and DNase may be included in the lysis solution, but not reagents that degrade mRNA.

Mechanical rupture of single cells has been described. Reference 5 discloses a method for fast lysis of a single cell (or cellular component thereof) by generating a shock wave, and to minimise manipulation trauma the cell is either positioned by laser tweezers or is cultured as an adhered cell. Ultrasonic vibration can also be applied to the device in order to lyse cells, as can laser light, which has previously been used to lyse single cells, as in reference 6. Lysis of single cells in a microfluidic device by osmotic shock is reported in reference 7. Reference 8 describes navigation and steering of single cells with optical tweezers to different areas of a microfluidic network where the flow properties can be controlled by electrophoresis and electroosmosis. A cell is captured between two electrodes where it can be lysed by an electric pulse.

Depending on the magnitude of the electric field used for electroporation, a membrane may simply be opened, allowing access to a cell's contents, or may rupture, leading to cell lysis (see reference 9). A field strong enough to cause lysis is preferred.

Before the samples are applied to the support, or after the samples have been applied to the support, it may be desirable to remove certain components from the samples and/or modify certain components of the samples. Biochemical analysis is often preceded by such purification or modification steps to remove substances which may interfere, either in terms of a target analyte's interaction with an analytical component, or in terms of accessing or interpreting results.

Protocols for preparing samples for analysis by microarray are well known in the art e.g. for cell disruption, for mRNA purification, for cDNA preparation, for genomic DNA purification, for polypeptide purification, for labelling, etc.

For example, if mRNA is the desired analyte for capture by an immobilised probe then DNA or protein may be removed before analysis. In the examples herein, it was found to be advantageous to use a multispecific protease composition to reduce non-specific signal derived from cellular proteins when analysing samples by reverse transcription of support-bound mRNA. Suitable sample processing steps will be evident to the skilled person, in light of the target analytes and samples to be analysed.

### Applying Samples to a Transfer Substrate

In some methods of the invention, the samples are applied to different areas of a transfer substrate to generate a spatial arrangement of samples, and then target analytes are transferred from the transfer substrate to a support. When a transfer substrate is used, the spatial arrangement of target analytes after transfer to the support matches the spatial arrangement of samples on the transfer substrate, thus permitting the results of the analysis to be matched to individual samples. The use of a transfer substrate can facilitate the initial generation of a suitable spatial arrangement of samples.

Samples can be applied to a transfer substrate by any suitable method, including but not limited to pipetting, printing, spotting and spreading. For example, samples can be applied to a transfer substrate using a sample applicator of the type described in U.S. design Pat. D 413,390.

The transfer substrate may be constructed of any suitable material. The choice of material for the transfer substrate is influenced by a number of design considerations, and suitable materials can readily be selected by the skilled person based on the requirements of a particular device. For example, the material(s) should be stable to the reagents applied to the transfer substrate during use, and compatible with the method(s) chosen for transferring target analytes to the support. In some embodiments, the transfer substrate may be made from nitrocellulose.

The transfer substrate can be substantially planar, e.g. a sheet material. The transfer substrate can be substantially non-planar, e.g. an initial spatial arrangement of samples can be generated on the pins of a spotter. Spotters are commonly used in the production of DNA arrays, and can readily be used in the methods of the invention. The individual pins of a spotter can be used to apply different individual samples to patches of different analytical components on a support. The individual pins of a spotter can also be used to apply different individual samples to a single patch on a support. An appropriate pin arrangement can be selected by the skilled person to complement the arrangement of patches on the support and the type of analysis required.

Transfer of target analytes from the transfer substrate to the support, while maintaining the spatial arrangement of the target analytes, can be achieved in a variety of ways. A suitable transfer method can be selected based on the specific transfer substrate material, samples and target analytes involved.

In some embodiments, transfer of target analytes is facilitated by contacting the support with the transfer substrate. In other embodiments, transfer of target analytes is facilitated by positioning the transfer substrate in close proximity to the support. The transfer substrate and/or support can be subjected to conditions which favour transfer of target analytes to the support. For example, a transfer reagent can be applied to the substrate and/or support. A transfer reagent is any reagent which can facilitate transfer of target analytes from the transfer substrate to the support.

The target analytes can be transferred from the transfer substrate to the support by a passive transfer method, such as diffusion, or by an active transfer method, such as by suction or electrokinesis. For example, the transfer substrate may be an electrically or magnetically conductive material, such that an electrical potential or a magnetic field may be applied to the transfer substrate and/or the support to facilitate transfer of target analytes from the substrate to the support.

In some embodiments, the transfer substrate is impermeable to target analytes. When the transfer substrate is impermeable to target analytes, samples can be applied to a surface of the transfer substrate and that surface then positioned against or in close proximity to the support for transfer of target analytes from the substrate to the support.

In some embodiments, the transfer substrate is impermeable to transfer reagents. In some embodiments, the transfer substrate is impermeable to target analytes and to transfer reagents.

In some embodiments, the transfer substrate is permeable to target analytes and transfer reagents. When the transfer substrate is permeable to target analytes and transfer reagents, transfer of target analytes from the transfer substrate to the support may involve movement of target analytes through or out from within the transfer substrate. For example, samples can be applied to a first side of the substrate to generate a spatial arrangement of samples on the substrate. A transfer reagent can then be applied to the substrate, such that target analytes are carried through the substrate to a second side of the substrate, from which second side they can be transferred to the support. For example, the transfer substrate can be a porous membrane and the transfer reagent can be a buffer.

Thus, when a transfer substrate permeable to a transfer reagent is used, the transfer reagent can be applied to the transfer substrate to cause transfer of target analytes from the transfer substrate to the support. Although transfer reagents are advantageous when used with a substrate permeable to the transfer reagent (and preferably, also permeable to the target analytes), transfer reagents can also be used with impermeable substrates. An appropriate transfer reagent can be selected by the skilled person, and will depend on the type of device to be use and the samples to be analysed. For example, the transfer reagent can be a buffer.

In some embodiments, it is not necessary for the entire sample to be transferred to the support for analysis. Indeed, in some embodiments, it may be preferable that only certain components of each sample are transferred, for example if the samples are complex samples (e.g. cells) that might contain undesirable interfering components.

Thus, in some embodiments, the transfer substrate is permeable to target analytes and transfer reagents, but impermeable to other components of the samples, such as cells or cell components. For example, the transfer substrate may be impermeable to whole cells, certain cell types, cell fragments, such as cell membranes, and/or organelles, etc. In those embodiments, samples comprising cells (or material derived from cells) can be applied to a first side of the transfer substrate, to generate a spatial arrangement of samples on the transfer substrate. A transfer reagent can then be applied to the substrate, such that target analytes are carried through the substrate to a second side of the substrate, from which second side they can be transferred to the support, without co-transfer of whole cells, certain cell types, cell fragments, organelles, etc. Thus, the transfer substrate may assist in sample preparation, by allowing transfer of target analytes to the support while preventing or reducing transfer of other components of the samples to the support.

In embodiments where cells are applied to the transfer substrate, the transfer reagent preferably also functions as a lysis reagent, so that the number of reagents required is minimised.

In some embodiments, the transfer substrate may specifically or non-specifically capture one or more components of the samples, other than the target analytes. Specific or non-specific capture of sample components may reduce the background signal caused by those components, and thereby improve the results of the analysis.

For example, if the target analyte is a protein, the transfer substrate may specifically or non-specifically capture nucleic acids. Specific capture of nucleic acids can be achieved using an immobilised binding reagent as described herein. Non-specific capture of nucleic acids may be achieved using a transfer substrate that adsorbs or absorbs nucleic acids but not proteins. For example, some positively charged Nylons are designed to adsorb nucleic acids.

For example, if the target analyte is a nucleic acid, the transfer substrate may specifically or non-specifically capture proteins. Specific capture of proteins can be achieved using an immobilised binding reagent as described herein. Non-specific capture of proteins may be achieved using a transfer substrate that adsorbs or absorbs proteins but not nucleic acids. For example, nitrocellulose adsorbs proteins and single stranded DNA, but not RNA or double stranded DNA.

In some embodiments, 50% or more (such as at least 60%, at least 70%, at least 80%, at least 85%, at least 90%, at least 95%, at least 97%, at least 98%, at least 99%, or at least 99.5%) of a specific target analyte, or of a specific type of analyte such as mRNA, in each sample is transferred from the transfer substrate to the support. Embodiments in which 85% or more (such as at least 90%, at least 95%, at least 97%, at least 98%, at least 99%, or at least 99.5%) is transferred permit more accurate quantitation of target analytes.

In some embodiments, less than 50% (such as less than 5%, less than 10%, less than 20%, less than 30%, or less than 40%) of a specific target analyte, or of a specific type of analyte such as mRNA, in each sample is transferred from the transfer substrate to the support. Such embodiments leave some of the target analytes on the support for subsequent manipulation by other methods, e.g. the PCR.

The fraction of a specific target analyte, or of a specific type of analyte such as mRNA, in each sample that is transferred from the transfer substrate to the support can be varied by varying the method used to transfer target analytes from the transfer substrate to the support appropriately. For example, the proximity of the transfer substrate to the support, the transfer reagent used, the strength of the electrical potential or the magnetic field, the temperature at which transfer occurs, and/or the time allowed for transfer, can be varied to provide the desired level of target analyte transfer.

It is also envisaged that an enzymatic reaction might be performed on the samples after application to the transfer substrate, but before transfer of target analytes from the transfer substrate to the support.

### The Spatial Arrangement of the Samples

In the methods of the invention, samples are applied to a support or a transfer substrate to generate a spatial arrangement of samples, and that spatial arrangement is maintained during the subsequent steps of the methods. The generation of a spatial arrangement of samples, in particular a spatial arrangement of target analytes, is a key feature of the present invention.

The generation of a spatial arrangement of samples in the methods of the invention is analogous to the generation of a spatial arrangement of analytes in other methods, such as Southern blotting. However, in the methods of the invention, a spatial arrangement of target analytes is generated on a support, to which an analytical component is immobilised. In contrast, in e.g. Southern blotting, a spatial arrangement of target analytes is generated on a support, and the analytical component is provided in the solution phase, such that different target analytes cannot readily be detected in parallel.

### Generating a Spatial Arrangement of Samples

A spatial arrangement of samples is initially generated when the samples are applied to a support or to a transfer substrate.

In some embodiments, samples are applied to the support or transfer substrate to generate a random spatial arrangement of samples (see FIG. 4A). For example, when the samples are biological samples comprising cells, a cell suspension can be appropriately diluted and then applied to the support or transfer substrate to generate a random spatial arrangement of cells. The spatial arrangement generated in such methods will resemble the spatial arrangement of cells observed during use of conventional hemocytometers. Random sample application methods do not require a pre-determined sample application pattern, and may be quicker to implement.

Random sample application methods may require the spatial arrangement of samples to be identified, so that the spatial arrangement of signal observed in the analysis step can be correlated with the spatial arrangement of the samples (see FIG. 5). Otherwise, it may not be possible for negative results to be identified in some situations. The spatial arrangement of samples on the support or transfer substrate may be visualised by any suitable method, such as specific or non-specific labelling (e.g. staining for protein or membrane components when the samples comprise cells). The spatial arrangement of samples on the support or transfer substrate may be recorded by any suitable method, such as digital image capture, if required. In the examples herein, the spatial arrangement of individual cells on a glass support was identified by brightfield microscopy or high-resolution laser scanning.

If identification of the spatial arrangement of samples is required, then the material(s) chosen for the support or transfer substrate should be compatible with the chosen identification method. For example, if the spatial arrangement is to be identified by digital image capture, a translucent or transparent support or transfer substrate may be preferred.

In some embodiments where samples are randomly applied to the support or the transfer substrate, it will not be necessary to identify the spatial arrangement of the samples. In particular, it may not be necessary to identify the spatial arrangement of the samples if an actual or average number of samples applied to each patch or to the device is known. For example, if in the FIG. 5 experiment it was known that 10 samples had been applied to the support, then when 5 signal spots are observed it may be concluded that half of the samples contained the target analyte. This type of statistical analysis is particularly useful when suspensions of cells are applied to a support or substrate, because the number of cells per unit volume of the suspension will generally be known. For example, if an average of 50 cells is applied to each patch on the support and only 5 signal spots observed on a particular patch, it may be concluded that approximately 10% of the cells contained the relevant target analyte.

In other embodiments, samples are applied to the support or transfer substrate to generate an ordered spatial arrangement of samples (see FIG. 4B). For example, when the samples each comprise the contents of a cell or a population of cells, the samples can be applied to the support or transfer substrate in a directed manner, e.g. using a printer or plotter, to generate a pre-determined spatial arrangement of samples on the support or transfer substrate. Non-random sample application methods may not require identification of the spatial arrangement of samples as described above (because it is pre-determined), and may also allow more samples to be applied to each patch because of more efficient use of the available space. In the examples herein, samples were applied to a support using a manual spotter (see Example 10)

Samples can be applied to the support or transfer substrate individually. Individual sample application is preferred when samples are applied to the support or transfer substrate to generate an ordered spatial arrangement of samples.

Samples can be applied to the support or transfer substrate in one or more groups of samples. Grouped sample application is preferred when samples are applied to the support or transfer substrate to generate a random spatial arrangement of samples.

Preferably, a sample is applied to only one patch of analytical component, such that a sample does not contact >1 patch on the device. However, in some embodiments it may be preferred for a sample to be applied to more than one patch of analytical component, such that a sample contacts >1 patch, such as 2 patches, 3 patches, 4 patches, or more.

### Maintaining the Spatial Arrangement of Target Analytes

After a spatial arrangement of samples is initially generated by applying the samples to a support or transfer substrate, the spatial arrangement of the target analytes in the samples will be maintained during the subsequent steps. The maintenance of a spatial arrangement of target analytes is a key feature of the present invention.

The reagents and materials used in the methods and devices of the invention should be selected to allow for maintenance of the spatial arrangement of target analytes. The spatial arrangement of target analytes will be affected by diffusion of the target analytes in three dimensions (i.e. both lateral and vertical diffusion) prior to capture by the analytical component. The amount of diffusion that occurs will depend on various factors such as proximity of the target analytes to the analytical component before capture, the temperature at which the device is used, the time between sample application and target analyte capture and the specific reagents used.

Thus, the devices and reagents of the invention may comprise components selected to minimise lateral and/or vertical diffusion of target analytes. For example, a dialysis membrane may be used to reduce vertical diffusion of target analytes away from the support, whilst allowing liquid reagents such as lysis buffer to be applied to the samples (see the examples herein). For example, sample preparations may contain additives selected to prevent lateral diffusion of target analytes (see the examples herein).

The sample manipulation and analysis steps in the methods of the invention may also be optimised to reduce diffusion of target analytes.

The spatial arrangement of samples is maintained during the methods of the invention, such that there is no significant movement of a sample relative to the other samples. Thus, there will be no significant change in the centre-to-centre separation of samples, even though there may be some spreading of target analytes during the methods of the invention, such that inter-sample spacing (edge-to-edge separation) is reduced. The spatial arrangement of samples is adequately maintained where the signal arising from one sample can be distinguished from the signal arising from a different sample, and the spatial arrangement of signal generated during the analysis step can be correlated with the initial spatial arrangement of samples. Generally, the two-dimensional arrangement of the samples is maintained, even if the three-dimensional arrangement of the samples is not maintained (e.g. the shape of individual cells is lost during lysis).

In some embodiments, the spatial arrangement of the target analytes is maintained such that there is no significant movement of target analytes relative to the immobilised analytical components. Thus, in some embodiments, the spatial arrangement of the target analytes in the samples is maintained such that there is no significant movement of a sample relative to the other samples, and such that there is no significant movement of samples relative to the immobilised analytical components. For example, in some embodiments the positions of the target analytes relative to the different patches on a support are maintained, i.e. there is no significant movement of target analytes across the support.

As noted elsewhere herein, the methods of the invention do not require differential labelling of different samples—the different areas of the support or substrate where individual samples are applied will be known or can be identified, so the signal generated by each individual sample can readily be identified. However, differential labelling might be useful in some embodiments of the invention. For example, differential labelling might be used to allow parallel analysis of samples derived from different sources (e.g. parallel analysis of individual cells in two different blood, or food, samples) using a single patch of analytical component. The use of differential labelling in conjunction with the invention may enable more information to the read from each patch of analytical component, but may also complicate analysis of the results.

In the methods of the invention, it is the spatial arrangement of the target analytes that will be maintained, rather than the spatial arrangement of all sample components. For example, the methods of the invention may comprise washing steps in which some sample components are lost from the device. In such methods, the spatial arrangement of the target analytes will be maintained, but the spatial arrangement of other sample components will not be maintained.

The generation and maintenance of a spatial arrangement of samples as described herein permits the results of the analysis to be matched to individual samples. However, it will not always be necessary for the results of the analysis to be matched to individual samples. The step of matching the results of the analysis to the individual samples is therefore optional. In some embodiments it will be sufficient to analyse the signal observed across a whole patch, for example by recording the average signal intensity for the patch. In other embodiments, it will be necessary for the results of the analysis to be matched to individual samples. For example, a device according to the invention could be used to detect the presence of a bacterium in a food sample by applying multiple cells from the food sample to a patch of analytical component, and recording the average signal for the patch. In that situation, the presence of the bacterium would be indicated qualitatively by the observation of a signal (or the observation of a increased signal relative to a negative control). The relative abundance of the bacterium in the sample cells could be determined by performing a more detailed quantitative analysis, if desired.

### Analysing Results

The detection methods used to analyse results depend on the nature of the target analyte and on any label that may be used. They may also depend on the strength of the signal at a given analysis site, as explained in more detail below. Detection methods used with DNA and protein microarrays and/or with membrane based methods are suitable for use in conjunction with the present invention; some such methods are described in more detail below.

The methods of the invention may involve qualitative and/or quantitative detection of the target analyte(s). Quantitative detection methods are preferred.

In some embodiments, analysing results will include correlating the spatial arrangement of signal generated with the spatial arrangement of samples (see FIG. 5). This correlation may be performed manually, but is preferably automated e.g. using image analysis software to compare the spatial arrangement of signal with the spatial arrangement of samples. The output of this correlation may be a composite image, in which both the spatial arrangement of samples and the spatial arrangement of signal are shown.

For the preferred analytes (mRNA and protein), further biochemical processing may be needed in order to introduce detectable labels after a target analyte has interacted with an immobilised binding reagent. Fluorescent labels are preferred for use with the invention. The fluorescence being detected preferably results from specific binding of two biological molecules e.g. two nucleic acids, an antibody & antigen, etc. Intercalating dyes may be used for detection of target analytes.

Fluorescence can be excited using an evanescent wave. These waves extend out of the surface of a material by ˜½ of the wavelength of the illuminating light i.e. they will extend outwards by ˜150-350 nm, which is more than enough to extend illumination throughout a patch of immobilised oligonucleotides. As mentioned elsewhere herein, a device of the invention may include a laser source (and/or a laser detector). Other sources of light for excitation can also be used e.g. lamps, LEDs, etc.

Proteins can be detected by one of several known methods that exploit antibodies. For example, a protein that has been captured by an immobilised antibody can be detected by applying a second labelled antibody specific for a different epitope from the first antibody, to form a ‘sandwich’ complex, or by using staining the protein.

For RNA analytes, detection can be achieved by incorporating fluorescent nucleotides into a complementary strand using an enzyme such as reverse transcriptase (e.g. the avian myeloblastosis virus (AMV) reverse transcriptase). For example, cDNA may be made in situ by hybridising mRNA to oligonucleotide probes on a support, and using the immobilised probe as a primer. The reverse transcription reaction preferably incorporates labelled nucleotides into the cDNA in order to facilitate detection of the hybridisation [10]. This can be achieved by the use of dNTPs with suitable fluorophores attached. Unlike a sequencing reaction, it is not necessary to use different coloured fluorophores for different nucleotides, because individual nucleotides do not need to be distinguished. Similarly, there is no need to label every nucleotide, and so 1, 2, 3 or 4 of dATP, dCTP, dGTP and dTTP may be labelled, and a mixture of labelled and unlabelled dNTPs can be used. Incorporation of a large number of fluorophores into the cDNA (e.g. in at least 5% of incorporated dNTPs, such as ≧10%, ≧20%, ≧30%, ≧40%, ≧50%, ≧75%, or more) means that the cDNA can readily be detected by any of the familiar means of fluorescence detection, thus revealing a positive signal even for a single hybridisation event. Thus even low-abundance mRNAs can be detected.

Rather than incorporate fluorophores directly, it is also possible to incorporate a specific functional group to which fluorophores can later be coupled (‘post-labeling’) e.g. after steps such as reverse transcription, washing, etc.

Sensitive techniques are available for detection of single fluorophores [11, 12], however, and so detection of an individual cDNA/mRNA hybrid containing multiple fluorophores is well within current technological capabilities. Current apparatuses that can identify single fluorophores have a pixel resolution of ˜150 nm. For example, references 13 & 14 describe a single molecule reader (commercially available as the ‘CytoScout’ from Upper Austrian Research GmbH) in which a CCD detector is synchronized with the movement of a sample scanning stage, enabling continuous data acquisition to collect data from an area 5 mm×5 mm within 11 minutes at a pixel size of 129 nm. In some of the examples herein, a proprietary high-resolution laser scanner was used to obtain 130 nm resolution data, and detection of single mRNA molecules was possible. Accordingly, in some embodiments, the methods, devices and kits of the invention allow detection of individual target analyte molecules, such as individual mRNA molecules.

After in situ reverse transcription has been performed, there is initially a RNA/DNA hybrid, wherein the DNA will typically include a label for detection. In some embodiments of the invention, the RNA strand in this hybrid is removed e.g. using RNAse H. This removal step leaves a single-stranded DNA, which has been prepared by extension of an immobilised primer. After the removal step, this single-stranded cDNA can be used as the template for synthesis of the complementary cDNA strand, thereby giving double-stranded cDNA. Synthesis of this second strand will be initiated using a primer that is complementary to the existing cDNA strand. After the initial reverse transcription, only DNA that had been extended as far as the location of this primer will be available for priming second strand synthesis. The second cDNA strand may also be synthesised to incorporate label, and the label can be the same as or different from the label used during synthesis of the first strand.

Target analytes bound to immobilised analytical components may also be amplified, for example by rolling circle amplification (RCA, e.g. references 15 and 16) or multiple displacement amplification (MDA; e.g. references 17 and 18). Suitable reagents are commercially available (e.g. from Qiagen Ltd., Crawley).

Target analytes bound to immobilised analytical components may also be detected by chemiluminescence methods. Suitable methods for detecting target analytes by chemiluminescence have been reported (e.g. references 19 and 20) and suitable reagents are commercially available (e.g. from Applied Biosystems, Foster City, Calif.). For example, reverse transcription of captured RNAs can be performed using biotinylated dNTPs, and the product detected by applying (strept)avidin-HRP or (strept)avidin-AP followed by a chemiluminescence substrate, and then image capture.

As mentioned elsewhere herein, a device of the invention can also be interfaced with a mass spectrometer. Integration of microfluidic devices with MS is known. For example, reference 21 describes a microfluidic chip for peptide analysis with an integrated HPLC column, sample enrichment column, and nanoelectrospray tip, and this ‘HPLC-Chip/MS Technology’ is available from Agilent.

Performing identical individual analysis in parallel on different cells is particularly powerful and readily allows differences to be detected in apparently identical cells.

The present invention permits quantitation of the proportion (e.g. percentage) of a set of cells that contain the target analyte.

Preferably, the number of samples that can be analysed in parallel for a given target analyte is at least 5 (e.g. ≧10, ≧15, ≧20, ≧25, ≧30, ≧35, ≧40, ≧45, ≧50, ≧60, ≧70, ≧80, ≧90, ≧100, ≧200, ≧300, ≧400, ≧500, ≧600, ≧700, ≧800, ≧900, ≧1000, etc).

### General

The term “comprising” encompasses “including” as well as “consisting” e.g. a composition “comprising” X may consist exclusively of X or may include something additional e.g. X+Y.

The term “about” in relation to a numerical value x means, for example, x±10%. Where necessary, the term “about” can be omitted.

The word “substantially” does not exclude “completely” e.g. a composition which is “substantially free” from Y may be completely free from Y. Where necessary, the word “substantially” may be omitted from the definition of the invention.

The use of terms such as “diameter” and “circumference” in relation to an element does not necessarily imply that the element is circular (or, in a three-dimensional context, spherical).

The term “antibody” includes any of the various natural and artificial antibodies and antibody-derived proteins which are available, and their derivatives, e.g. including without limitation polyclonal antibodies, monoclonal antibodies, chimeric antibodies, humanized antibodies, human antibodies, single-domain antibodies, whole antibodies, antibody fragments such as F(ab′)₂ and F(ab) fragments, Fv fragments (non-covalent heterodimers), single-chain antibodies such as single chain Fv molecules (scFv), minibodies, oligobodies, dimeric or trimeric antibody fragments or constructs, etc. The term “antibody” does not imply any particular origin, and includes antibodies obtained through non-conventional processes, such as phage display. Antibodies of the invention can be of any isotype (e.g. IgA, IgG, IgM i.e. an α, γ or μ heavy chain) and may have a κ or a λ light chain.

### BRIEF DESCRIPTION OF THE DRAWINGS

FIG. 1 illustrates schematically the general approach when only a single analytical component is used.

FIG. 2 illustrates schematically the general approach when different analytical components are immobilised in different patches on a support.

FIG. 3 illustrates schematically the general approach of the invention when a transfer substrate is used.

FIG. 4 illustrates generation of random (FIG. 4A) and non-random (FIG. 4B) spatial arrangements of samples.

FIG. 5 illustrates how a spatial arrangement of signal can be correlated with a spatial arrangement of samples.

FIG. 6 illustrates schematically the device used in Example 1 herein.

FIG. 7 shows the scanned images for the two slides used in Example 1 herein.

FIG. 8 shows in more detail regions of the two slides used in Example 1 herein.

FIG. 9 illustrates schematically the device used in Example 2 herein.

FIG. 10 shows the scanned images for the two slides used in Example 2 herein.

FIG. 11 illustrates schematically the device used in Example 3 herein.

FIG. 12 shows in detail a region of the slide used in Example 3 herein.

FIG. 13 shows a region of the slide used in Example 5 herein.

FIG. 14 shows a region of the slide used in Example 5 herein.

FIG. 15 shows a region of the slide used in Example 5 herein.

FIG. 16 shows in more detail a region of the slide used in Example 5 herein.

FIG. 17 shows in more detail a region of the slide used in Example 5 herein.

FIG. 18 shows in more detail a region of the slide used in Example 5 herein.

FIG. 19 shows a region of the slide used in Example 8 herein.

FIG. 20 shows a region of the slide used in Example 9 herein.

FIG. 21 shows the slide used in Example 10 herein.

FIG. 22 illustrates schematically a possible device comprising a permeable support.

FIG. 23A illustrates schematically the probe application pattern used in Example 12.

FIG. 23B shows the results of the 10 second exposure in Example 12.

FIG. 24A illustrates schematically the probe application pattern used in Example 13.

FIG. 24B illustrates schematically the sample application pattern used in Example 13.

FIG. 24C illustrates schematically the device used in Example 13.

FIG. 25A shows a scanned image for the slide used in Example 13 after hybridisation, but before reverse transcription.

FIG. 25B shows a further scanned image for the slide used in Example 13 after hybridisation and reverse transcription.

FIG. 25C shows a further scanned image for the slide used in Example 13 after hybridisation, reverse transcription and mixing in SDS solution overnight.

FIG. 26 illustrates schematically the sample application pattern used in Example 14.

FIG. 27 shows the results of the different exposures in Example 14.

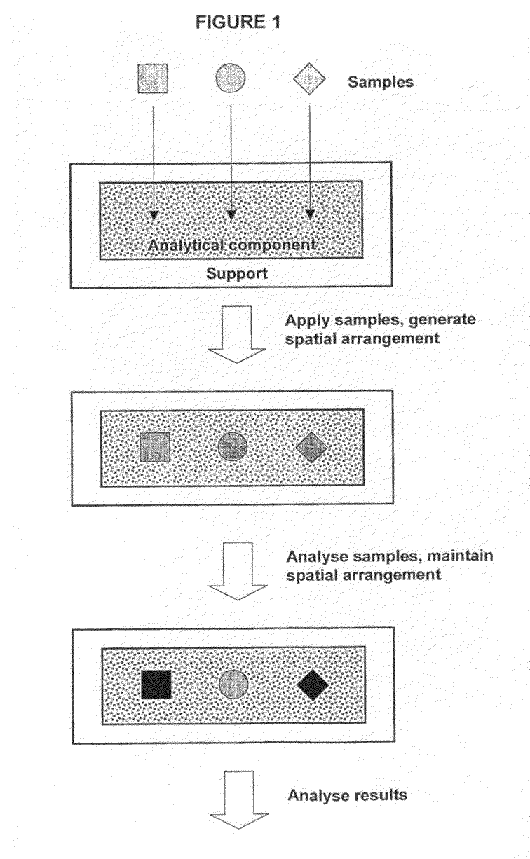

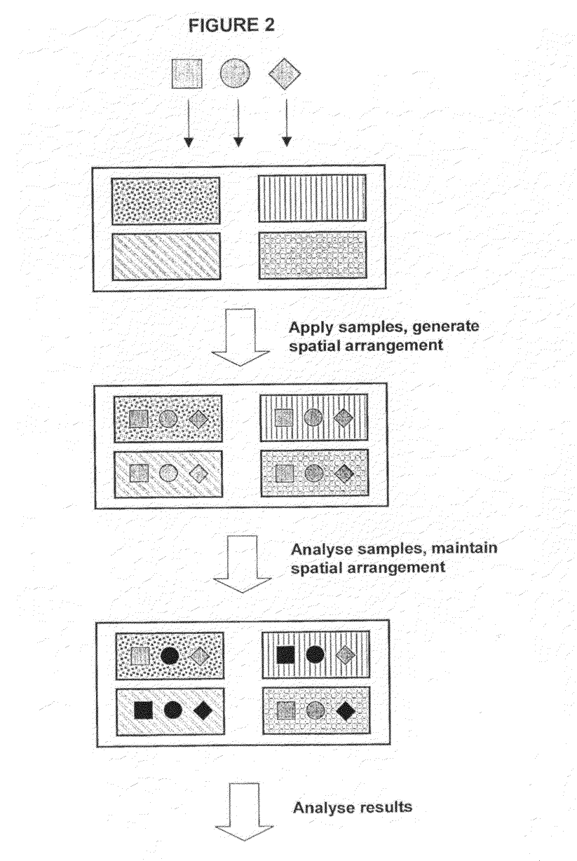

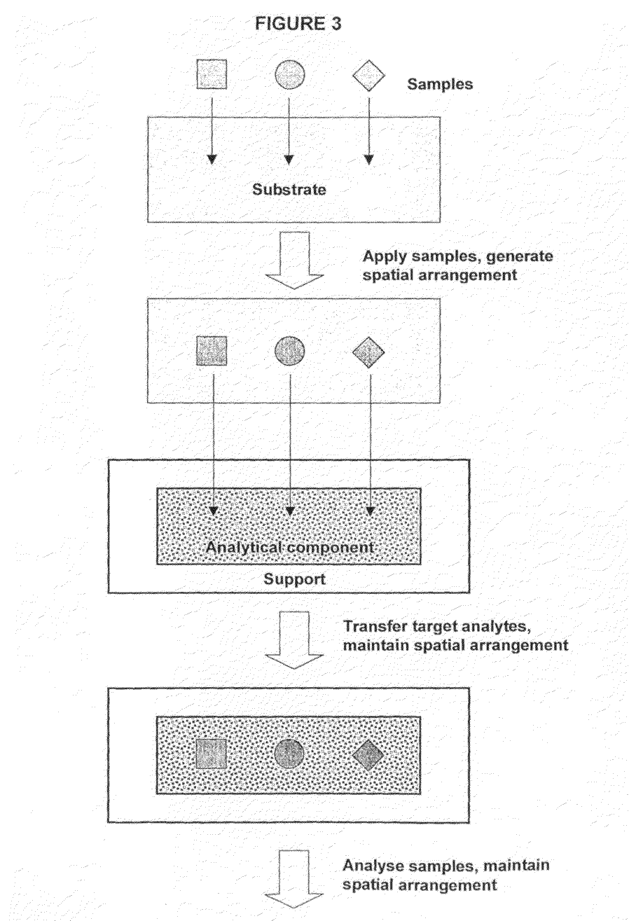

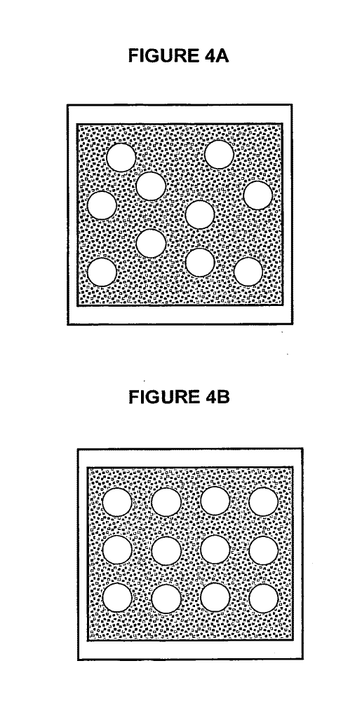

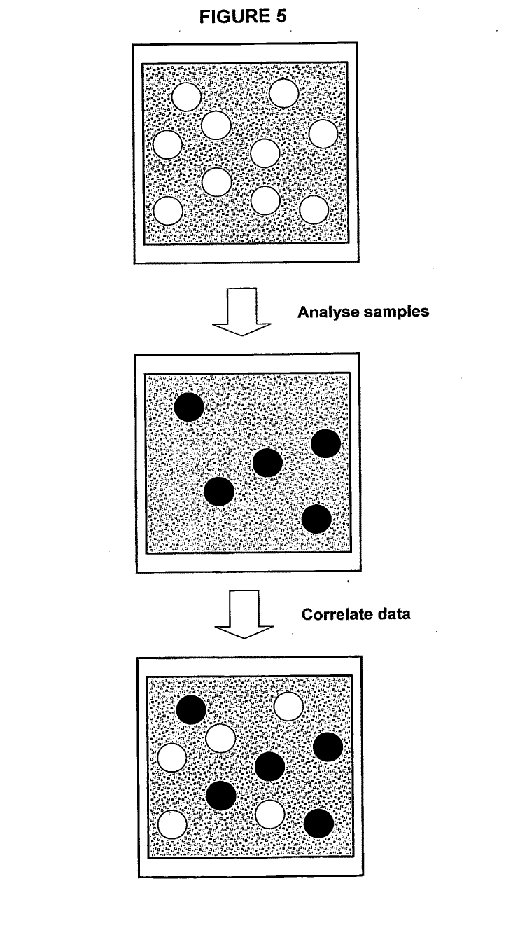

### MODES FOR CARRYING OUT THE INVENTION

### Example 1

### Analysis of Individual Cells

Preliminary experiments were performed to confirm that individual samples, in particular individual cells, can be applied to a support to generate a spatial arrangement of samples, and that the spatial arrangement of samples can be maintained during the subsequent manipulation and analysis steps.

### Materials and Methods

Oligo dT₃₀ glass slides were prepared by adding 1 μl (100 μM) oligo dT₃₀ to 100 μl of a 1:1 mix of phosphate buffer (pH 9.0):DMSO. The oligo and coupling buffer mix was applied to an NHS derivatised slide (Schott) using a 21 mm×40 mm×0.1 mm Hybriwell chamber (Sigma). The oligo was allowed to couple to the slide for 15 minutes. The Hybriwell chamber was removed and the slide washed for 5 mins in distilled water. Negative control slides were prepared by 3′ attachment of oligos.

A mouse myeloma suspension (non-adherent) cell line was used. A suspension of 3000 cells/μl was prepared by repeated centrifugation and washing in 1×PBS. The cells were mixed with PEG 2000 at 20% and PEG 200 at 20%. PEG 200 was used to prevent clumping of cells due to the hydrophobic nature of the support surface. PEG 2000 was used to prevent lateral diffusion by polymer exclusion.

In some experiments, pronase was added to the cell suspension at 1 mg/ml. Pronase is a mixture of endo- and exo-proteinases, that is capable of cleaving almost any peptide bond.

0.5 μl of the cell suspension was spread onto the slide, and then dried. Cells with pronase in the cell suspension were spread on the left hand side of the slides. Cells without pronase in the cell suspension were spread on the right hand side of the slides.

Polyacrylamide gel pads of 100 mm×100 mm×1 mm 10% polyacrylamide (19:1) in water were poured and allowed to set. Rectangles of gel about 5 mm×5 mm were cut and soaked in 1% SDS lysis buffer (1% SDS, 3×SSC) for at least 30 minutes.

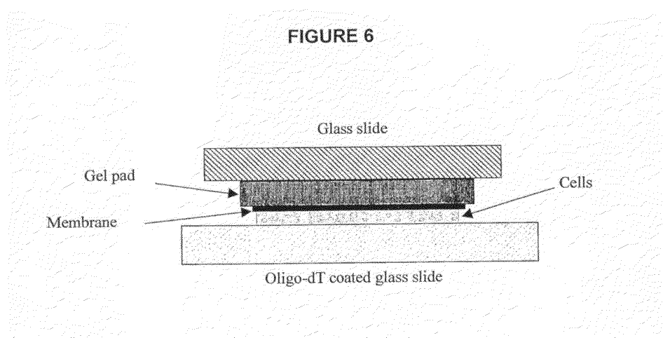

A dialysis membrane was placed against the sample cells to minimise vertical diffusion of target analytes. A gel pad was placed against the cellulose nitrate membrane, allowing the lysis buffer to diffuse through the membrane to contact the cells. A glass slide was placed on top of the gel pad, to create good liquid contact between the gel pad, membrane, and sample cells. The assembled device is schematically illustrated in FIG. 6.

The assembled device was incubated at 50° C. for 30 minutes to aid pronase digestion of cellular proteins. The device was then incubated at room temperature for 30 minutes, to allow for hybridisation of cellular mRNA to the oligo dT binding reagent. After the incubation steps, the slides were washed and bound mRNA reverse transcribed in a 100 μl reaction volume. A Hybriwell chamber was applied to each slide and incubated at 50° C. before application of the reverse transcription mix. 100 μl reverse transcription mix contained: water (63.6 μl), 5×FS buffer (20 μl), RNasin (1 μl), 0.1M DTT (10 μl), 25 mM dNTP mix (0.4 μl), CY3 dCTP (1 μl), Superscript III enzyme (4 μl). The reaction was incubated at 50° C. for 30 mins. The slides were then washed and scanned with an Agilent G2565BA scanner.

### Results

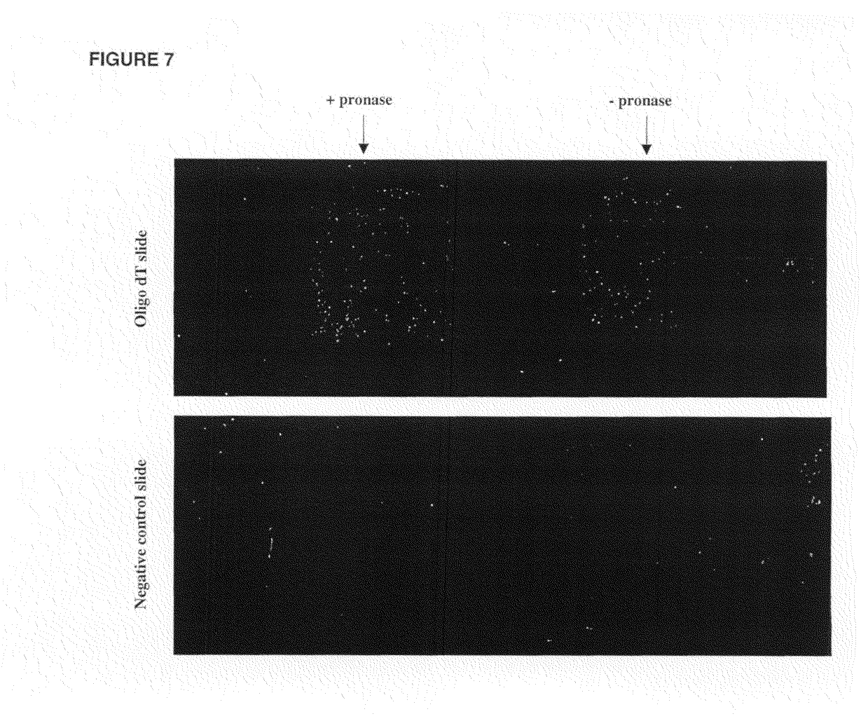

The scanned images for the oligo dT and negative control slides are shown in FIG. 7.

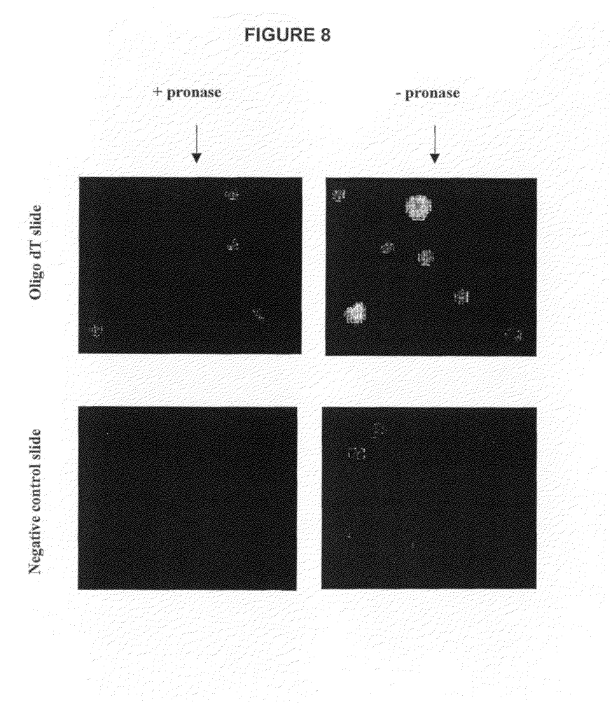

Looking at the right hand side of the oligo dT slide in FIG. 7, a large number of spots are seen that apparently correspond to signal from individual cells. However, spots are also visible on the right hand side of the negative control slide in FIG. 7, which suggests that some of the spots are due to non-specific signal. It was postulated that non-specific signal might arise from interaction of the dye with cellular proteins. Accordingly, in a second series of experiments, cells treated with pronase were analysed. Looking at the left hand side of FIG. 7, it can clearly be seen that the addition of pronase provides a significant reduction in the non-specific signal observed. Regions from the left and right hand sides of the two slides are shown in more detail in FIG. 8. That figure further illustrates that the addition of pronase provides a significant reduction in the non-specific signal observed.

The features seen on the oligo dT coated slide for the pronase-treated cells in FIG. 8 are likely to be specific signal derived from primer extension of oligo-bound mRNAs. Evenly sized images of 8-10 pixels wide (40-50 μm wide) were observed, with an intensity consistent with detection of transcripts in single cells of 10 μm with limited spreading of the cell contents.

### Conclusions

These experiments illustrate that a spatial arrangement of cells can be generated on a support, and the cells analysed whilst maintaining the spatial arrangement of cells. These experiments suggest that, for analysis of specific cellular target analytes such as mRNA, removal of other sample components, such as proteins, may be necessary to reduce non-specific signal.

### Example 2

### Timecourse of Lysis

Another series of experiments was performed to investigate the effect of varying the timecourse of lysis on the observed signal. Devices similar to those described in Example 1 were used.

A mouse myeloma suspension (non-adherent) cell line was used, as in Example 1. The cell suspension for these experiments contained 3000 cells/μl in 20% PEG 2000, 20% PEG 200 and 1 mg/ml pronase. 3 μl of the cell suspension was spread onto an oligo dT slide, dried and covered with a dialysis membrane. Two identical oligo dT slides were prepared, and one was used as a negative control by omitting the reverse transcriptase enzyme. On each of the two oligo dT slides, cells were lysed for 20 mins, 55 mins, 1 hr 30 mins or 1 hr 45 mins using four separate polyacrylamide gel pads as in Example 1. After the incubation steps, the slides were analysed as described in Example 1 by reverse transcription and scanning.

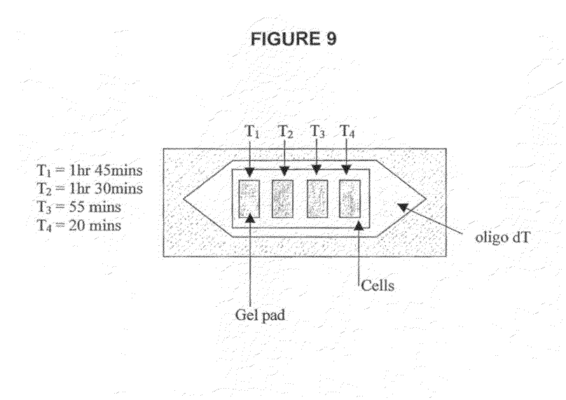

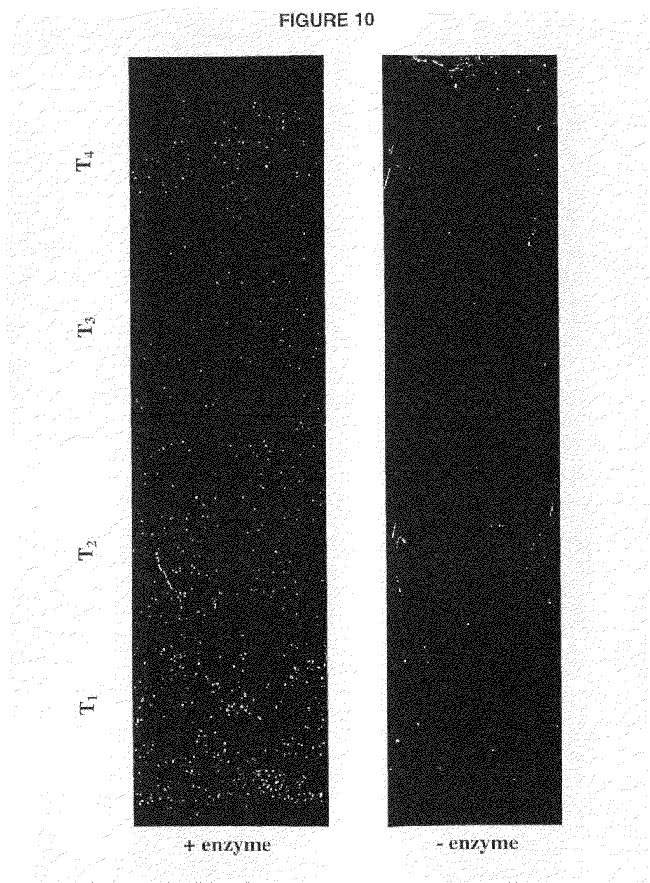

The assembled device is schematically illustrated in FIG. 9. The scanned images for the two slides are shown in FIG. 10.

For the slide with reverse transcriptase, the signal intensity was found to diminish as the lysis time reduces, except that the observed signal after 20 mins lysis was higher than that after 55 mins lysis. This is likely to be due to non-specific signal arising from incomplete pronase digestion of cellular proteins after 20 mins.

Very little signal was observed across the control slide, to which no reverse transcriptase had been added.

Thus, these experiments suggest that longer incubation times will be preferred, to allow complete cell lysis, target analyte capture and digestion of cellular proteins.

### Example 3

### Use of Collodion

In this experiment, the dialysis membrane used in the device of Example 1 was replaced with a thin layer of collodion. Collodion is a solution of nitrocellulose in ether or acetone, sometimes with the addition of alcohols, and is generically referred to as pyroxylin solution.

100 μl of collodion was pipetted onto one end of the slide and then spread over the whole slide. After the incubation step, the slide was washed, the collodion removed by washing the slide in acetone containing a small amount of MgCl₂, and the bound mRNA reverse transcribed as described elsewhere.

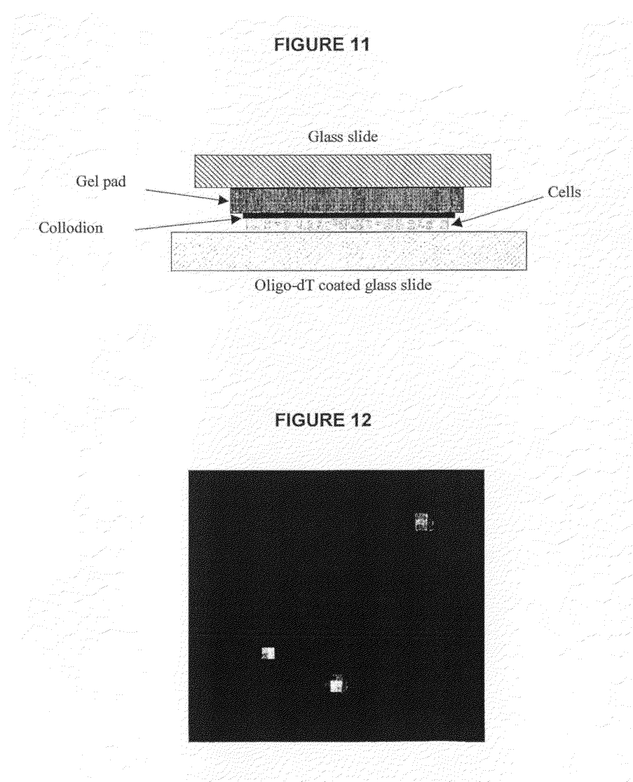

The assembled device used in this experiment is schematically illustrated in FIG. 11. The image captured for an area of the slide in shown in FIG. 12. In this experiment, the images arising from individual cells were relatively small and collodion appears effective in preventing the lateral spread of cellular contents. In particular, in FIG. 12, the images are 20-25 μm for a 15 μm cell size, indicating that little spreading of cell contents has occurred.

### Example 4

### Other Experiments

Other experiments have also been performed to investigate factors affecting the generation and maintenance of the spatial arrangement of cells, the detection of target analytes and the reduction of false positives.

In one experiment, a polyacrylamide gel pad was applied directly to cells that had been spread onto the surface of the slide and air dried. In another experiment, cells were mixed with molten low Tm agarose and spread as a thin layer onto the surface of the slide, and then a polyacrylamide gel pad was applied directly to the agarose. In another experiment, cells were spread onto a cellulose nitrate membrane, the side of the membrane to which the cells were applied placed on the surface of the slide, and a polyacrylamide gel pad placed on the other side of the membrane. The results of those experiments also confirm that a spatial arrangement of cells can be generated on a support, and the contents of the cells analysed whilst maintaining the spatial arrangement of cells.

In some experiments, Triton lysis buffer (320 mM sucrose, 5 mM MgCl₂, 10 mM Hepes buffer, 1% Triton X-100, 0.2% Trypan blue stain) was used in place of the SDS lysis buffer. Triton lysis buffer was found to produce results roughly equivalent to those produced when SDS buffer was used.

### Example 5

### Further Analysis of Individual Cells

This example demonstrates that a spatial arrangement of individual cells can be generated on a support, the spatial arrangement of cells identified, and the spatial arrangement of signal observed in the analysis step correlated with the spatial arrangement of the individual cells.

### Materials and Methods

A glass slide coated with 5′-immobilized oligo dT₃₀, as in Example 1, was used. A mouse myeloma suspension cell line was used, as in Example 1. In this experiment, cells were fixed to the slide using 80% MeOH. The slide was pre-warmed (to 65° C.). The MeOH evaporates quickly from the pre-warmed slide, fixing the cells to the surface of the slide with minimal clumping. Approximately 50,000 cells were pipetted onto the slide. No PEG 2000 or PEG 200 was used in this experiment. After fixing to the slide, the cells were covered with 10 mg/ml pronase, either by coating (pipetting and spreading) or by aerosol spraying.

The slide was then scanned in a proprietary high-resolution laser scanner (130 nm pixel resolution), as described in United Kingdom patent applications GB 0618131.7 and GB 0618133.3.

The cells were then lysed in SDS lysis buffer using a polyacrylamide gel patch (as in Example 1), and the released mRNA captured on the support (1 hr at 50° C.). Captured mRNA was then reverse transcribed from the immobilised oligo dT₃₀ primers, as previously. The slide was then scanned again using the same high-resolution laser scanner, to identify fluorescent reverse transcription products.

### Results

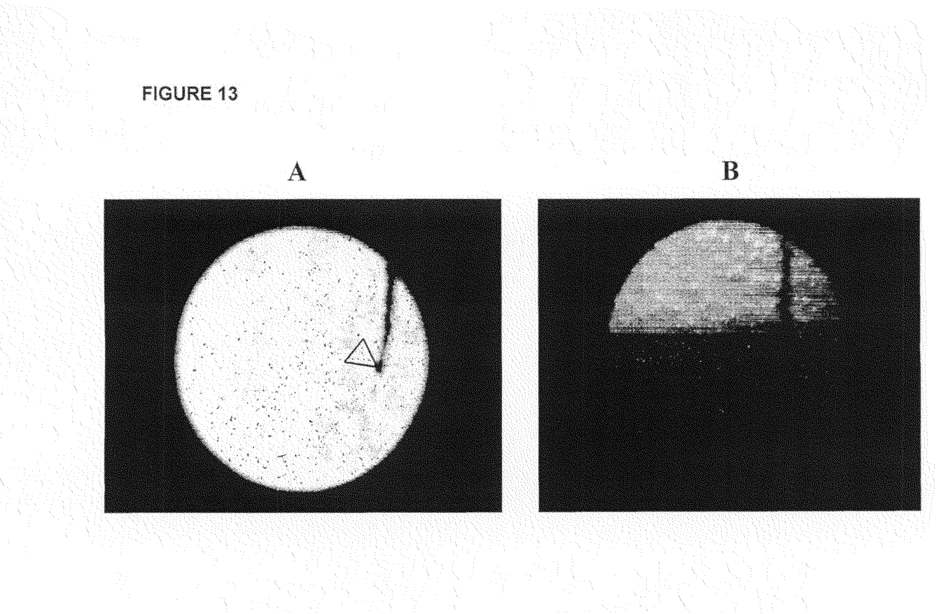

FIG. 13 illustrates the spatial arrangement of cells on the slide before (FIG. 13A) and after (FIG. 13B) pronase treatment, as viewed by brightfield microscopy. Triangular alignment guides are shown, to aid comparison of the cell locations. As highlighted by those alignment guides, the spatial arrangement of cells on the slide is maintained after pronase treatment. FIG. 13 also illustrates that brightfield microscopy can be used to identify the spatial arrangement of individual cells on a support.

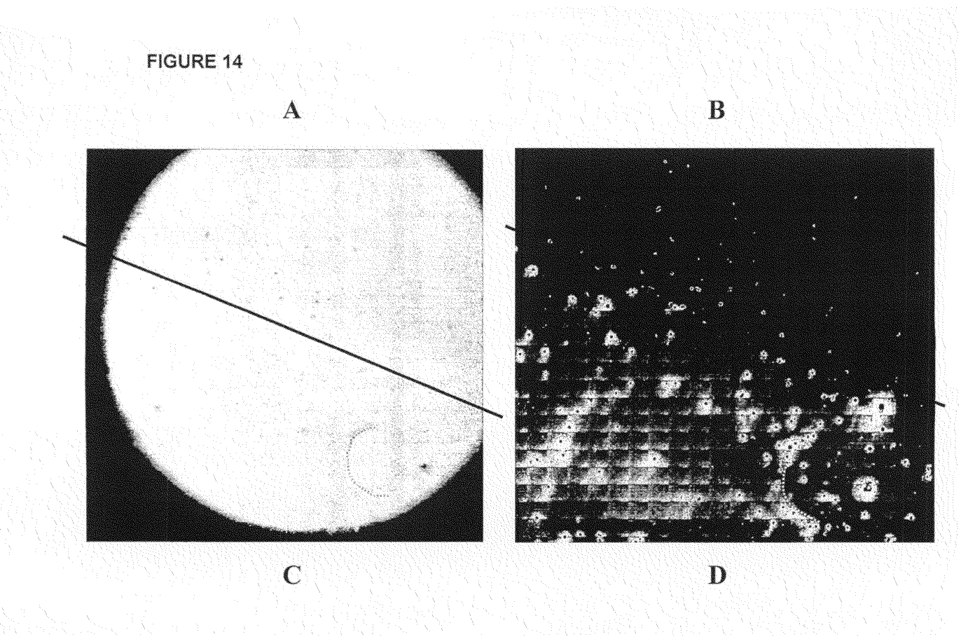

FIG. 14 shows the spatial arrangement of individual cells on the slide with and without pronase treatment, as viewed by brightfield microscopy (FIGS. 14A and C) or high-resolution laser scanning (FIGS. 14B and D). The region above the diagonal line was not treated with pronase (FIGS. 14A and B). The region below the diagonal line was treated with pronase (FIGS. 14C and D). Semicircular alignment guides are shown in FIGS. 14C and D, to aid comparison of the cell locations. As highlighted by those alignment guides, the spatial arrangement of cells on the support is maintained after pronase treatment and laser scanning. After addition of pronase, individual cells are clearly visible due to autofluorescence (FIG. 14D). Thus, addition of pronase facilitates identification of the spatial arrangement of individual cells by autofluorescence, as well reducing non-specific signal (see Example 1).

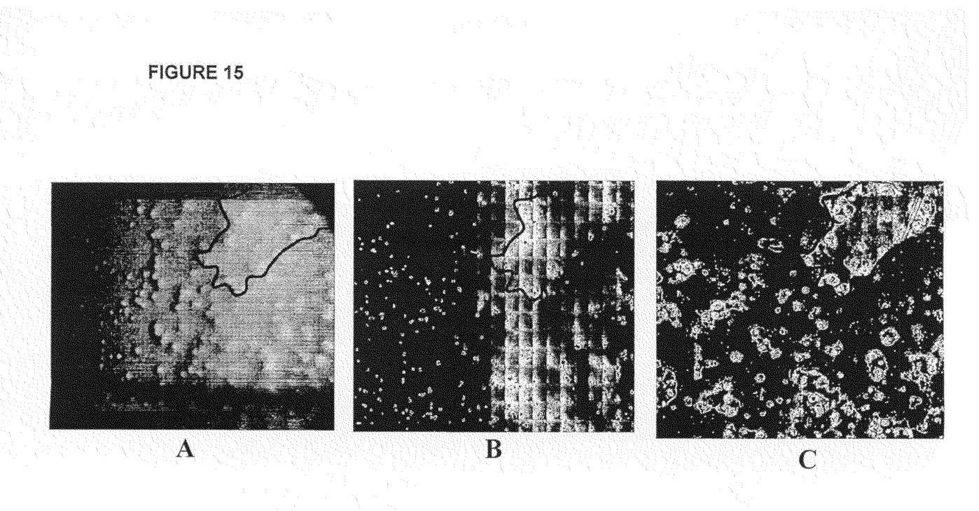

FIG. 15 shows the spatial arrangement of individual cells on the support, as viewed by brightfield microscopy (FIG. 15A) or high-resolution laser scanning (FIGS. 15B and C). FIG. 15B shows autofluorescence of pronase-treated cells, whereas FIG. 15C shows the fluorescence signal observed after reverse transcription of captured mRNA. Alignment guides are shown in FIG. 15, to aid comparison of the cell locations. As highlighted by those alignment guides, the spatial arrangement of mRNA on the support is maintained during reverse transcription, such that the spatial arrangement of the fluorescence signal observed in FIG. 15C can readily be correlated to the spatial arrangement of individual cells in FIGS. 15A and B.

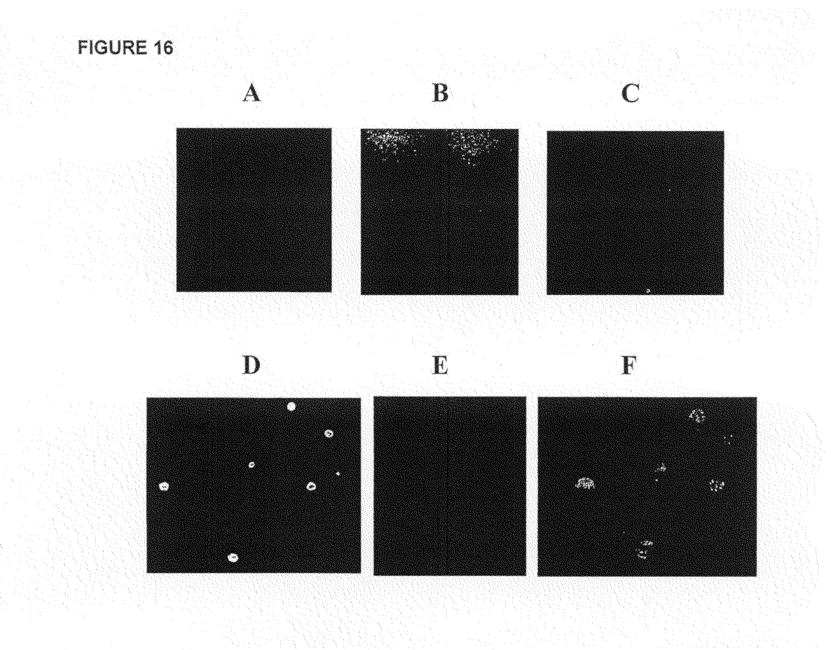

FIG. 16 shows a more detailed view of a region of a slide at each stage of the protocol used in this example. FIG. 16A shows the blank glass slide before oligo dT₃₀ attachment. FIG. 16B shows the slide after oligo dT₃₀ attachment. FIG. 16C shows the oligo dT₃₀-coated slide after cells were fixed. FIG. 16D shows the slide after treatment with pronase. FIG. 16E shows the slide after cells were lysed. FIG. 16F shows the slide after reverse transcription of immobilised mRNA. As illustrated by this series of images, the addition of pronase is responsible for the observed autofluorescence of fixed cells (in particular, see FIG. 16D). The autofluorescence of pronase-treated cells is lost after cell lysis (FIG. 16E).

As illustrated by FIG. 16, a spatial arrangement of individual cells can be generated on a support, the spatial arrangement of cells identified, and the spatial arrangement of signal observed in the analysis step correlated with the spatial arrangement of the samples.

### Conclusions

These results reinforce the results in Examples 1-4, and further confirm that a spatial arrangement of individual cells can be generated on a support, the spatial arrangement of cells identified, and the spatial arrangement of signal observed in the analysis step correlated with the spatial arrangement of the individual cells.

### Example 6

### Analysis of Target Analyte Spreading

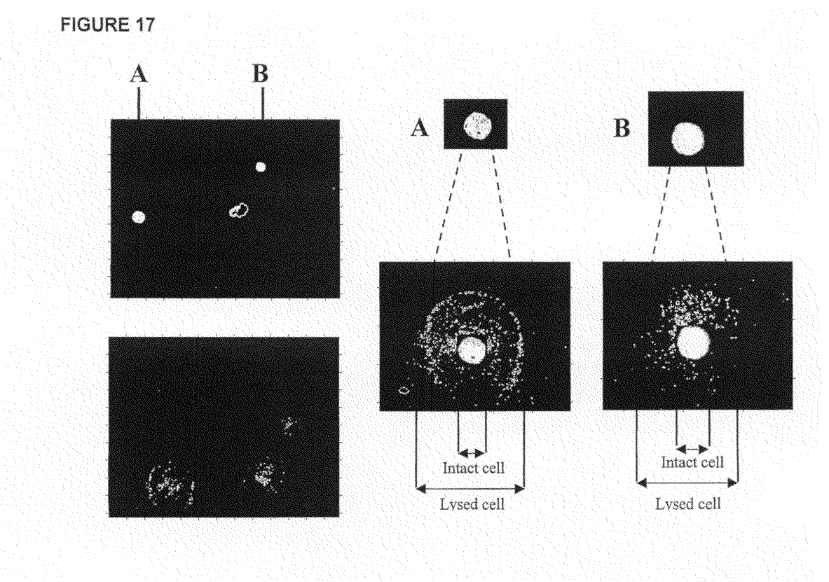

This example investigates target analyte spreading after cell lysis. FIG. 17 shows a detailed analysis of the fluorescence signal for two cells (A and B) observed in Example 5. As illustrated in that figure, there is a 3-4 fold increase in the sample footprint following cell lysis (from 15-20 μm to 50-80 μm). These results suggest that the minimum area required by each individual cell, to prevent target analyte overlap after lysis, is approximately 100 μm². Smaller minimum areas will be possible following further optimisation of the invention.

### Example 7

### Detection of Single mRNA Molecules

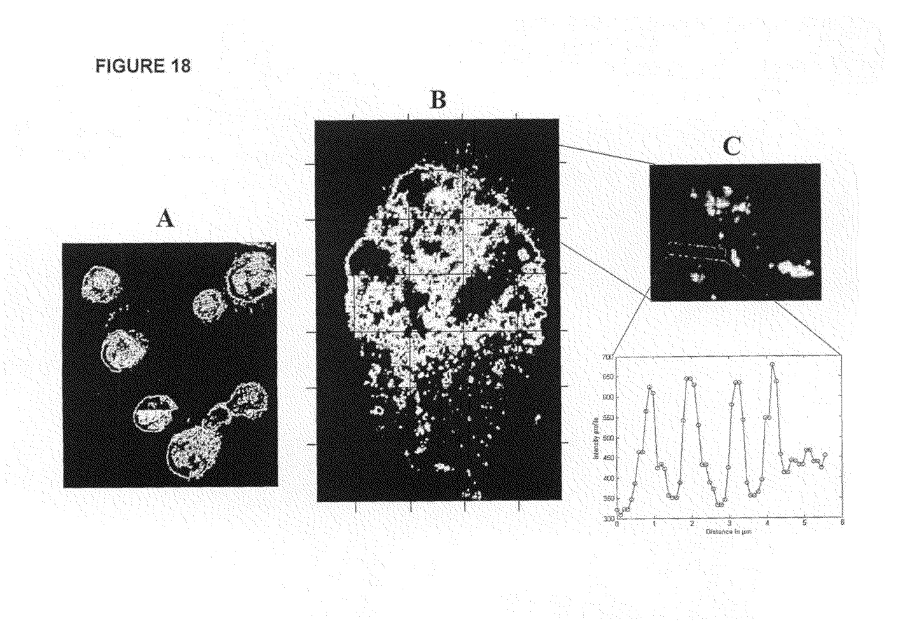

This example demonstrates that the methodology in Example 5 permits detection of single mRNA molecules when the support is scanned with a high-resolution laser scanner (130 nm pixel resolution). FIG. 18A shows the fluorescence signal observed in a 250 μm×250 μm region of the support—the individual cells can be distinguished. FIGS. 18B and C show a detailed analysis of the fluorescence signal observed following reverse transcription of mRNA from an individual cell. FIG. 18B shows the fluorescence signal observed from a single cell, with a 10 μm grid overlaid. FIG. 18C shows a region of single molecule resolution within FIG. 18B. As highlighted by the fluorescence intensity plot in FIG. 18C, a series of four individual fluorescent molecules were observed with approximately 1 μm spacing. Those observed signals result from reverse transcription of four individual mRNA molecules. Accordingly, the methods of the invention permit the detection of single mRNA transcripts. This is particularly useful for detection of specific mRNAs using gene-specific analytical components (see Example 8 below).

### Example 8

### Detection of Specific mRNAs

In this example, the methodology of Example 5 was followed, except that instead of coating the slide with oligo dT₃₀, the slide was coated with 50-mer oligonucleotides specific for the Arbp housekeeping gene. Accordingly, in this example the support was used to detect specific transcripts, rather than total cellular mRNA. FIG. 19A shows the fluorescence signal observed in a 250 μm×250 μm region of the support. The signals observed from individual cells are circled in FIG. 19A. FIG. 19B shows detection of gene-specific reverse transcription from single cells, with a 10 μm grid overlaid. This example demonstrates that the methods of the invention can be used for detection of specific mRNAs using gene-specific analytical components.

### Example 9

### Calculation of Optimal Sample Density

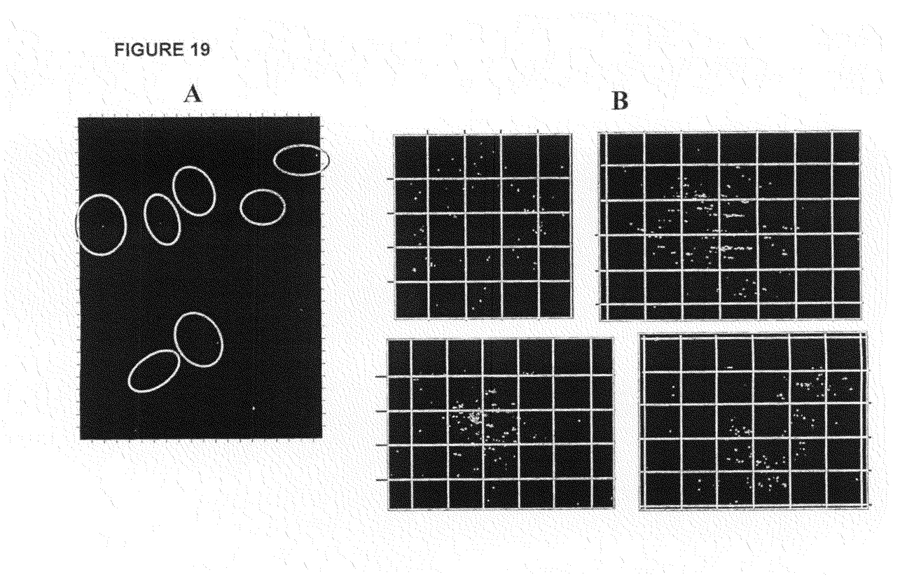

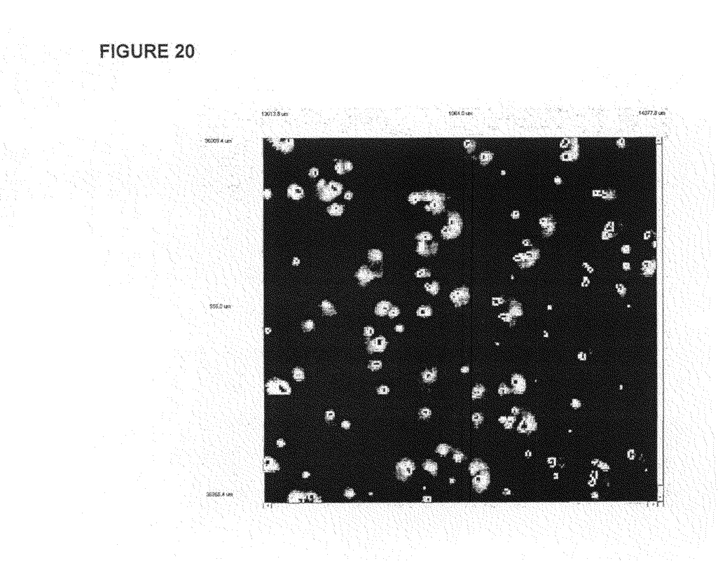

FIG. 20 shows an autofluorescent cell map for murine myeloma cells fixed to a glass slide after cells were loaded at a density of 500 cells/mm². After fixing the cells and high-resolution laser scanning (as in Example 5), the autofluorescent cell map reveals 111 cells/mm². This analysis suggests that the maximum cell loading density, when following the methodology in Example 5, should be approximately 100 cells/mm².

### Example 10

### Sample Spotting

In the previous examples, individual cells were applied to a support to generate a random spatial arrangement of cells. In this example, RNA samples were spotted at known positions onto large patches of oligonucleotide probes, to generate a non-random spatial arrangement of samples.

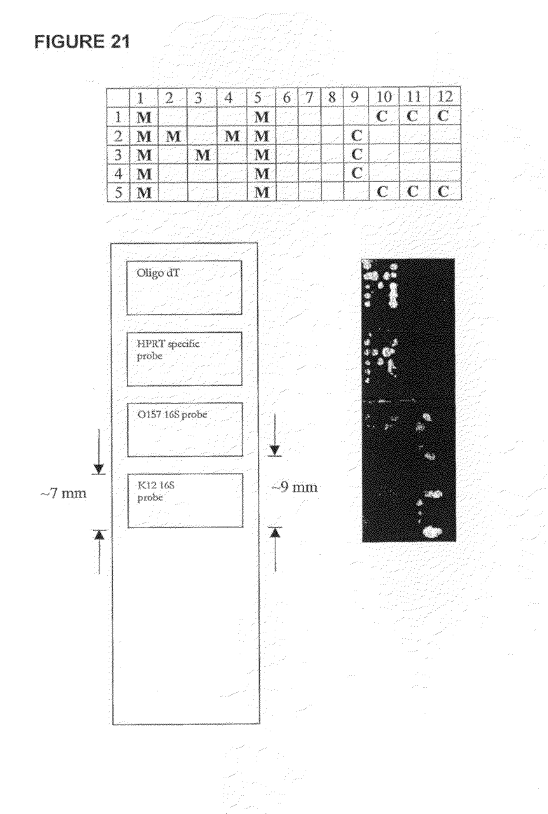

Oligonucleotides complementary to polyA (i.e. oligo dT), mRNA for HPRT and for the 16S ribosomal RNAs of E. coli strains K12 and 0157, with 5′—NH₂ termini, were coupled to a NHS ester derivatised glass slide in the pattern shown in FIG. 21, by applying solutions of the oligonucleotides under cover slips.

RNA extracted from cultured mouse lymphoblasts and from E. coli strain K12 were dissolved in 3×SSC at a concentration of ˜1.5 mg/ml. 1 μl of each RNA solution was pipetted into two wells of a 384 well microtitre plate. The samples were applied to the patches of probes using a Schleicher and Schuell manual spotter, with a pin spacing of 9 mm. The mouse RNAs were applied over all four patches in the pattern of a letter ‘M’ and the E. coli RNAs in the pattern of a letter ‘C’ as shown in FIG. 21. The solutions were allowed to dry at room temperature and the slides were chilled to −20° C. They were then washed and cDNA was synthesised in situ as described in Example 5, incorporating Cy3 labelled dCTP. The scan of the slide (FIG. 21) shows that the mouse RNA is specifically captured and reverse transcribed on the oligo dT and HPRT patches and the E. coli RNA on the 16S oligonucleotide patches.

Thus, this example demonstrates that samples can be applied non-randomly to a support to generate a non-random spatial arrangement of samples, and that the spatial arrangement of the samples can be maintained during the subsequent manipulation and analysis steps.

### Example 11

### Amplification on Membranes

In some embodiments envisaged by the inventors, materials permeable to the reagents used during use of the device are used to construct the support. Such supports may be advantageous in some embodiments, because they allow reagents to be passed through the support, which may facilitate cell capture, cell lysis, target analyte capture and/or analysis of target analytes.

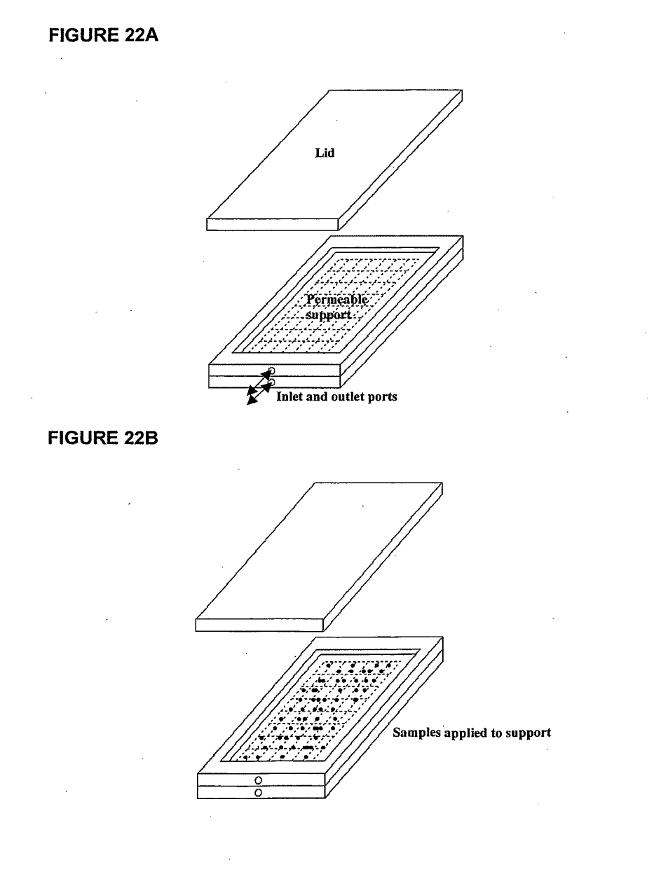

A possible arrangement is illustrated schematically in FIG. 22A, in which the device comprises a permeable support (with immobilised analytical components) disposed within a chamber formed in the device. The device may further comprise one or more inlet and/or outlet ports for adding and/or removing reagents. The use of inlet and/or outlet ports facilitates application of reagents to, and removal of reagents from, the support. In FIG. 22A, the device contains two such ports, and the permeable support is disposed within the chamber such that one port communicates with a first face of the support, and the other port communicates with a second face of the support. This allows one port to be used as an inlet port (i.e. to apply reagents to the support) and the other port to be used as an outlet port (i.e. to remove reagents from the support). Using this type of arrangement samples and reagents can easily be applied to, and removed from, the device (e.g. by injection or suction).

The device in FIG. 22A also comprises a lid, which can be used to keep the reagents within the device during use. The lid can be integral to the device, but may also be removable (as in FIG. 22A and FIG. 22B) to allow easy detection of target analytes. If the device is to be used for detection by fluorescence, then the lid and/or other parts of the device may be transparent to the excitation and emission wavelengths used for fluorescence detection, and may also have low intrinsic fluorescence at these wavelengths.

In the arrangement shown in FIG. 22, reagents can be applied to the permeable support through an inlet port, and removed from the device through an outlet. For example, a suspension of cells can be applied to the device, and the cells captured on the permeable support material (FIG. 22B). A lysis solution can then be applied to lyse the captured cells and to allow hybridisation of target analytes from individual cells to different areas of the support. The presence of target analytes in different individual cells can then be analysed by suitable methods.

To investigate detection methods that might be used in conjunction with devices comprising permeable supports, experiments were performed to determine whether DNA trapped in a membrane can be amplified by ‘Multiple Displacement Amplification’ (MDA, Qiagen). Preliminary experiments were performed to show that the polymerase enzyme used in MDA is not inhibited by the membrane.

E. coli K12 DNA was prepared from cells grown overnight in 15 ml L-broth. The cells were collected by centrifugation, resuspended in 1 ml PBS. 50 μl lysozyme (10 mg/ml) was added. Cells were collected by centrifugation, resuspended 300 mM NaOAc, made to 2% SDS and kept at 60° C. for 30 min. After 1× extraction with phenol and 2× with chloroform, the aqueous layer was drawn off and the DNA spooled after the addition of 1 volume i-propanol. After a wash in 80% EtOH, the DNA was dissolved 100 μl TE. The theoretical yield of DNA is 25 μg. The E. coli K12 DNA stock was 1 mg/ml.

The stock was diluted 2.5:10 in REPLI-g denaturing solution (Qiagen) and REPLI-g neutralising solution (Qiagen). 0.2 μl of the DNA solution was applied to Nylon and cellulose nitrate strips (˜1 mm×6 mm). MDA Master Mix (MM) was made up according to the supplier's instructions (Qiagen). Approximately 15 μl of MM was applied to each strip and the strips incubated at 30° C. in a moist chamber. The Nylon strips appeared dry after about 1.5 hrs. Water was added to both Nylon and cellulose nitrate strips.

Other related experiments were also performed in which membrane strips (˜1 mm×6 mm) loaded with 0.2 μl E. coli DNA mix were immersed in 5 μl of MM in tubes. Control tubes contained MM with no DNA or no membrane. The amplification reactions were stopped at 16 hrs and 68 hrs by adding 1 μl of stop solution to 4 μl of the solution. The reaction products were applied to a 1% agarose gel. The strips that had been moistened with the mix were inserted into the gel loading slot.

The results suggest that cellulose nitrate inhibits amplification, perhaps by adsorbing the enzyme, whereas Nylon does not inhibit the reaction. Single-stranded DNA bound to Nylon is amplified in high yield, and much of the amplified product remained on the Nylon.

This example demonstrates that individual DNA molecules can be amplified in situ on a membrane, which should enable very sensitive detection methods to be used in conjunction with permeable substrates. Amplification in situ of captured target analytes should enable a wide range of useful applications, e.g. bacterial typing from single copy genes, rather than ribosomal RNAs, comparative genomic hybridization (CGH), single nucleotide polymorphism (SNP) detection and single molecule sequencing.

### Example 12

### Reverse Transcription on Membranes

As noted above, in some embodiments envisaged by the inventors, materials permeable to the reagents used during use of the device are used to construct the support. To further investigate detection methods that might be used in conjunction with devices comprising permeable supports, experiments were performed to investigate reverse transcription of RNA hybridised to probes immobilised on permeable supports.

### Materials & Methods

Probes complementary to the 3′ end of the mRNA for HPRT and to the 16S ribosomal RNA of E. coli strain K12, each with 5′-NH₂ termini, were used. In these experiments, the HPRT-End probe acts as the positive control, and the C1 probe as the negative control. The probe sequences were:

HPRT-End
5′ [AminoC6] TTT TTT TTT TTT TTT TTT TTT TTT TTT
TTT AAT TTT TAG CAT TTA TTT ATT TGC ATT TAA AAG GA
3′ (65mer)
C1 probe complementarvyto K12 16S rRNA
5′ [AminoC6] TTT TTT TTT TTT TTT TTT TTT TTT TTT
TTT TGT TCC CGA AGG CAC ATT CT 3′ (50mer)

Seven different permeable support materials were tested: Polyamide, Nylon, Nitrocellulose, Durapore GVHP, Immobilon PSQ, Immobilon P and Immobilon FL. Each membrane material was cut to approx 1 cm×2.5 cm in size. The PVDF membranes (GVHP, Immobilon PSQ, Immobilon P and Immobilon FL) were wet in methanol first and then in TE buffer (10 mM Tris/HCl and 1 mM EDTA), whereas the other membranes were wet directly in TE buffer. The PVDF membranes were pre-wetted with methanol because they are extremely hydrophobic, and will not wet in aqueous solutions unless pre-wetted. The membranes were then placed on tissue wetted in TE buffer, and the probes applied.

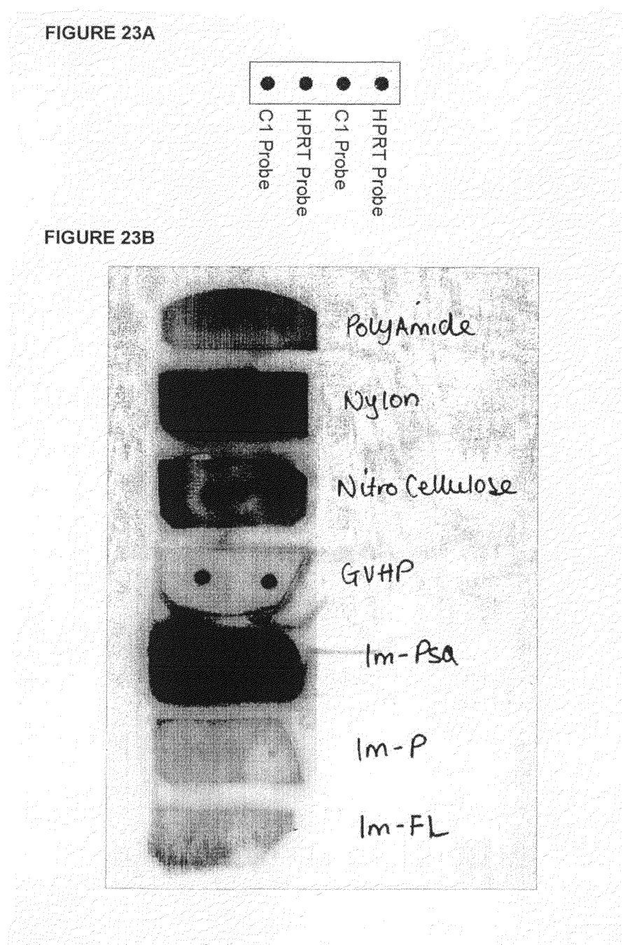

In particular, 5 μM concentrations of the two probes in TE buffer were made up, then 2×0.2 μl of each probe applied onto each of the seven membranes in the pattern shown in FIG. 23A. The probes were then crosslinked to the membranes for 2 minutes using a Stratalinker crosslinker (the Stratalinker was warmed for 10 minutes, then the membranes placed in the Stratalinker on damp tissue). After crosslinking, the membranes were placed in 15 ml Falcon tubes (3 or 4 membranes per Falcon) and washed with 5×SSPE/0.5% SDS for 30 mins in a rotating incubator at 55° C.

The target applied to the probes in these experiments was an unlabelled in vitro transcript (IVT) of the mouse HPRT mRNA of approximately 1250 bases in length. The HPRT IVT was prepared using the Epicentre AmpliCap T7 High Yield Messenger Maker kit. After in vitro transcription, Qiagen RNeasy MinElute Spin Columns were used for RNA clean up.

20 ml of hybridisation buffer (reference 22) was made up in a 50 ml Falcon tube. 10.2 μl of unlabelled HPRT IVT was added to the 20 ml of hybridisation buffer (2 ug/10 ml). The 5×SSPE/0.5% SDS was then removed from each Falcon tube, and 10 ml of the hybridisation buffer added to each Falcon tube. The tubes were placed in a rotating incubator for 1 hour at 45° C. After incubation, the hybridisation buffer was removed and 10 mls of 1×FS buffer added to each Falcon tube (5×=250 mM Tris-HCl, 15 mM MgCl2 and 375 mM KCl). The Falcon tubes were then placed on a rolling shaker until ready for reverse transcription (˜10 mins).

Reverse transcription was performed using the avian myeloblastosis virus (AMV) reverse transcriptase. 500 μl of reverse transcription mix was made up as follows: 428 μl water, 50 μl 10×AMV reverse transcriptase reaction buffer (New England Biolabs, 1×=50 mM Tris-HCl, 75 mM potassium acetate, 8 mM magnesium acetate and 10 mM DTT), 5 μl RNasin, 5 μl Biotin dUTP, 2 μl 25 mM dNTPs, and 10 μl AMV reverse transcriptase (New England Biolabs, M0277S 10000 units/ml). The membranes were removed from the Falcon tubes and each membrane placed into a small plastic wallet, sealed on three sides with a heat seal (i.e. they were left open on one side). 70 μl of the reverse transcription mix was added to each membrane, then the wallets were sealed using a heat sealer, and placed in an incubator at 42° C. for 1 hour.

After incubation, the membranes were removed from the plastic wallets and placed into Petri dishes (all 7 membranes in one Petri dish), then 25 ml wash buffer (reference 22) was added, and the membranes washed for 5 mins on a shaker. The wash buffer was then removed and replaced with a second wash buffer (reference 22), and the membranes washed for a further 5 mins. The second wash buffer was then removed and replaced with PBS/0.1% Tween, and the membranes washed for 5 mins. The PBS/0.1% Tween was replaced with fresh PBS/0.1% Tween, and then the membranes were washed for a further 5 mins.

Streptavidin-horseradish peroxidase was attached to the membranes. 2% blocking buffer was made up (from the ECL Advance Western Blotting Detection Kit: GE RPN2135) in PBS/0.1% Tween (1 g in 50 mls), and kept at 4° C. until required. The PBS/0.1% Tween was removed and replaced with 25 ml of blocking buffer per Petri dish. The dishes were then shaken for 1 hour. To the other 25 mls of blocking buffer 5 μl of ECL streptavidin horseradish peroxidase conjugate (from GE: RPN1231) was added, then the old blocking buffer removed and 25 ml of the blocking buffer containing the streptavidin added. The membranes were allowed to incubate in the streptavidin-HRP solution for 1 hr on a shaker. The solution was then discarded and the membranes washed in 25 ml PBS/0.1% Tween for 15 minutes on a rolling mixer. The PBS/0.1% Tween was then discarded, and replaced with fresh PBS/0.1% Tween and the membranes washed for a further 15 mins on the rolling mixer. The membranes were now ready for chemiluminescence detection.

Chemiluminescence detection was performed with ECL Advance. The detection reagents were allowed to equilibrate to room temperature before opening. 1 ml of detection solution A was mixed with 1 ml of detection solution B (from the ECL Advance Western Blotting Detection Kit: GE RPN2135). Excess wash buffer was drained off membranes, and the membranes placed flat on a piece of saran wrap, oligo side up. 100 μl of the mixed detection solution was applied onto each of the wet membranes, then the membranes incubated for 5 mins at room temperature. Excess detection solution was drained off by holding the membranes with forceps and touching the edge against a tissue. The membranes were then placed oligo side down onto a piece of plastic, the plastic folded over and sealed so that the membranes were totally flat and enclosed in the plastic. Care was taken to smooth out any air bubbles before sealing.

Detection was performed using Biomax XAR film. The sealed membrane was placed onto one half of a cassette, oligo side up. The cassettes were taken to a darkroom, and one film placed on top of the seven membranes, exposed for 10 seconds then removed. The film was placed in developing solution (from SIGMA, 500 mls 1 in 5 dilution with water) for 1 min, then rinsed in water for 5-10 s. The film was placed in fixing solution (from SIGMA, 500 mls 1 in 5 dilution with water) for 1 min. The film was then removed and washed thoroughly in water, and allowed to dry overnight by hanging. This process was repeated for 5 seconds and 30 second exposure times.

### Results and Conclusions

The results of the 10 second exposure are shown in FIG. 23B. The results for the 5 second and 30 second exposure times were substantially the same. These results suggest that reverse transcription works well using the AMV reverse transcriptase on nitrocellulose and GVHP, and works to a lesser extent using Immobilon-P and Immobilon-FL. These results thus confirm that RNA molecules can be reverse transcribed in situ on a membrane, which should enable sensitive detection methods to be used in conjunction with permeable supports. The results also confirm the finding in Example 11 that when a permeable material is used to construct the support, the choice of a particular support material may influence the detection method that should be used. These experiments further confirm that target analytes hybridised to analytical components immobilised on a permeable support may be detected by chemiluminescence methods, whilst maintaining the spatial arrangement of the target analytes.

### Example 13

### Use of a Transfer Substrate

As noted above, in some embodiments envisaged by the inventors, the samples are first applied to a transfer substrate to generate a spatial arrangement of samples, and then target analytes are transferred from the transfer substrate to the support. To further investigate the use of a transfer substrate, experiments were performed to investigate bacterial cell lysis on a transfer substrate, followed by target analyte transfer from the substrate to the support.

### Materials & Methods

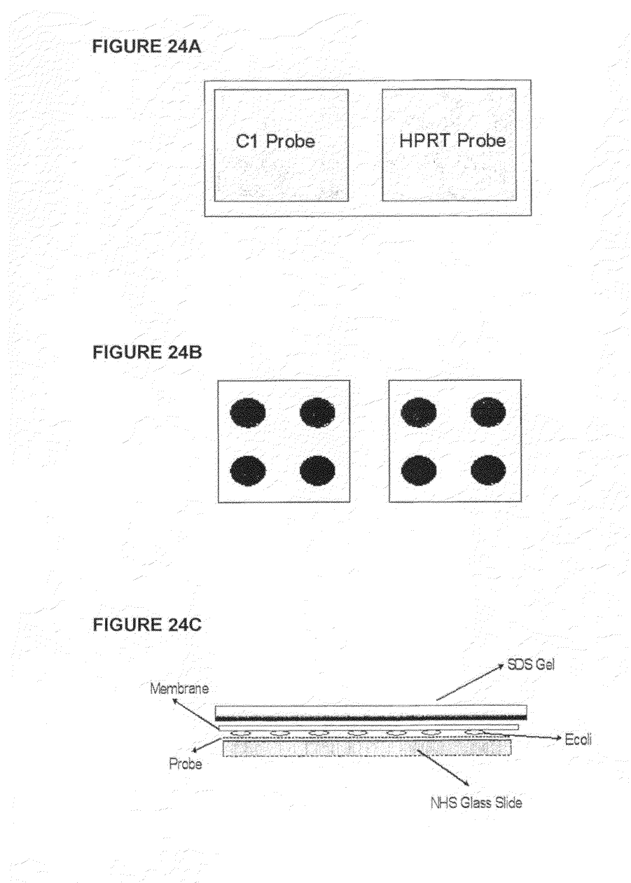

In these experiments, the support was a glass slide, which was used for hybridisation and reverse transcription of 16S rRNA from E. coli strain K12, followed by detection of CY3 cDNA. The support was designed as shown in FIG. 24A, with one patch of C1 probe and one patch of HPRT probe. These experiments used the same probes as in Example 12, but this time the C1 probe was used as the positive control, and the HPRT probe as the negative control.

A glass NHS derivatised slide (Schott) was taken out of the freezer and allowed to warm up to room temperature before removal from its case. 10 μl C1 oligo (100 uM) was diluted in 90 μl 1:1 0.2M K phosphate buffer (pH 9): DMSO to give a final oligo concentration of 10 uM. 10 μl HPRT oligo (100 uM) was diluted in 90 μl 1:1 0.2M K phosphate buffer (pH 9): DMSO to give a final oligo concentration of 10 uM. A lifter slip was cut in two and placed on top of the slide with coverslips as spacers between the NHS derivatised slide and the lifter slip. 100 μl of 10 μM C1 oligo was applied underneath the lifter slip on the left. 100 μl of 10 μM HPRT oligo was applied underneath the lifter slip on the right. The slide was left at room temperature for 30 minutes to allow the oligos to couple to the slide. At the end of coupling, the slide was placed in a Petri dish of water to deactivate the rest of the surface. The slide was placed in a Falcon tube with water and left washing for 30 mins on a rolling mixer.

SDS/Polyacrylamide gels were prepared as follows. A casting jig was prepared with two large microscope slides and two small microscope slides, using bulldog clips to hold it together. The gel mix (1 ml water, 625 μl acrylamide, 500 μl 10% SDS, 375 μl 10×SSC buffer, 30 μl 10% AMPS and 5 μl TEMED) was prepared, then applied to the casting unit, ensuring no bubbles. The gel was left to set for approximately 30 minutes. The gel was removed from the casting unit just before use.

E. coli cells were prepared for RNA hybridisation as follows. 3 ml of cells were added to 7 ml of LB media, then placed in an incubator at 37° C. for ˜2 hours. 1 ml of the prepared culture was taken into a 1.5 ml Eppendorf tube and 125 μl of cold 5% phenol in ethanol added. The contents of the tube were mixed by inverting to kill the E. coli, and then spun at 8.8 rpm for 2 mins. The supernatant was removed and 1 ml of 1×PBS added. The cells were resuspended by aspiration, then spun at 8.8 rpm for 2 mins. The supernatant was removed and 1 ml of 1×PBS added. The E. coli were again resuspended by aspiration. The resuspended cells were spun at 8.8 rpm for 2 mins, and the supernatant again removed. These steps remove any trace phenol.

200 μl of 0.5 mg/ml lysozyme (10 μl of 10 mg/ml stock+190 μl 1×PBS buffer) was added, then the E. coli cells resuspended by aspiration. The mixture was left at room temperature for 3-5 minutes to allow cell wall digestion. The cells were now ready to be applied onto the membrane. Two pieces of nitrocellulose membrane were cut to a size that would fit onto the slide. The membranes were placed in hot PBS for 5-10 minutes (the PBS was heated to 90° C. in a waterbath). The membranes were removed from the hot PBS and placed on a piece of tissue that had been soaked in PBS. 4×1 μl of cells were applied onto each of the membranes, in the pattern shown in FIG. 24B. 2 μl 10 mg/ml pronase solution was applied on top of each sample on the membranes.

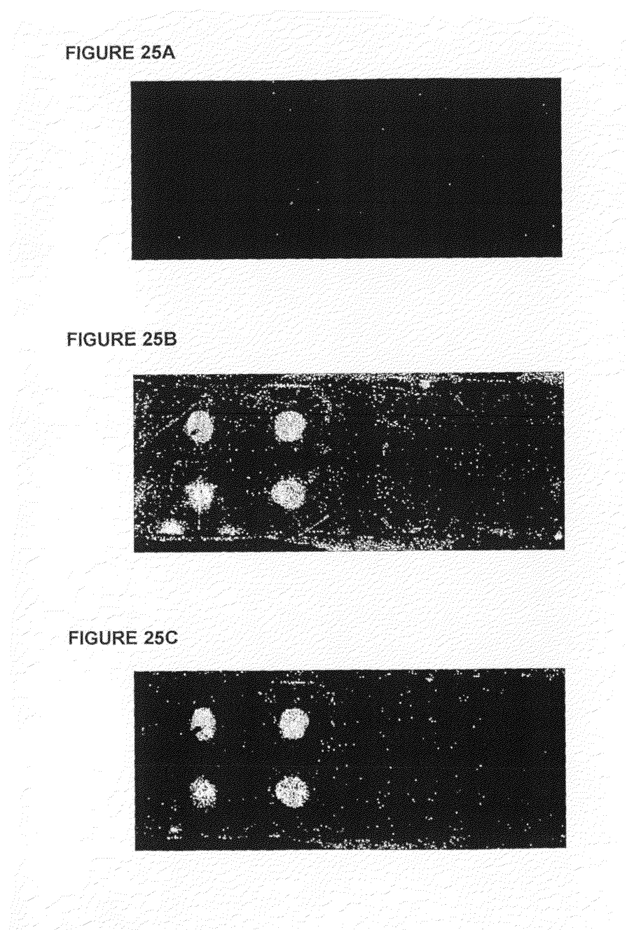

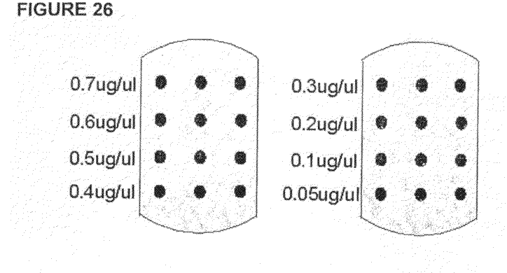

The slide was placed on a hot block at 45° C. The prepared membranes were applied onto the slide face down, so that the cells were directly on the probe patches. The membranes were covered with the casted polyacrylamide gel and a blank microscope slide placed on top of the gel, as shown in FIG. 24C. The assembly was left for 30 minutes to allow hybridisation. The slide was then removed from the hot block, and the gel pad and membrane removed. The slide was placed in a 50 ml falcon tube containing a wash buffer (reference 22) and placed on a rolling mixer, and washed for 5 minutes. The slide was then transferred into 50 ml of a second wash buffer (reference 22) and placed on a rolling mixer, and washed for a further 5 minutes. The slide was scanned using the Axon GenePix 4000B scanner (see FIG. 25A).

The captured 16S rRNA was reverse transcribed using CY3-dCTP. 250 μl of RT mix was made up (water 159 μl, 5×FS buffer 50 μl, RNasin 2.5 μl, 0.1M DTT 25 μl, 25 mM dNTP mix 1 μl, CY3-dCTP 2.5 μl, Superscript III enzyme 10 μl). The slide was placed on the hot block at 45° C. and a HybriWell chamber applied on top. 250 μl of RT mix was applied onto the slide, then the top of the HybriWell placed on the slide. The slide was incubated at 45° C. for 30 minutes. The HybriWell chamber was removed from the slide, and the slide placed in a Falcon tube. 50 ml of a wash buffer (reference 22) was added and mixed on a rolling mixer for 5 minutes. The slide was transferred into a fresh Falcon tube and 50 ml of a second wash buffer (reference 22) added, then mixed on a rolling mixer for 5 minutes. The slide was then scanned again using the Axon scanner (see FIG. 25B). The slide was placed in 1% SDS solution overnight on a rolling mixer, then scanned again using the Axon GenePix 4000B scanner (see FIG. 25C).

### Results and Conclusions

As is evident from FIGS. 25A-C, a fluorescence signal from CY3 cDNA was observed only at those areas of the support corresponding to the areas on the nitrocellulose membrane where the E. coli cells were applied. The pattern in which the cells were applied to the transfer substrate (FIG. 24B) is mirrored by the fluorescence signal pattern (FIGS. 25A-C). Furthermore, a fluorescence signal from CY3 cDNA was observed only at those areas of the support on which the C1 probe was present, confirming that the signal is indeed from reverse transcription of the E. coli 16S RNA. In other words, the pattern in which the probes were applied to the transfer substrate (FIG. 24A) is also mirrored by the fluorescence signal pattern (FIGS. 25A-C).

These experiments thus confirm that samples can be applied to a transfer substrate to generate a spatial arrangement of samples, and then target analytes transferred from the transfer substrate to a support, whilst maintaining the spatial arrangement of the target analytes. These experiments also confirm that the methods and devices of the invention can be applied to both eukaryotic and prokaryotic cells.

### Example 14

### Further Investigation of Membrane Chemiluminescence

Further experiments were performed to investigate use of chemiluminescence detection in conjunction with a permeable support material.

### Materials & Methods

Two Nylon membranes were cut to fit onto a microscope slide. The membranes were placed in a Petri dish and wet with TE buffer. The membranes were placed onto damp tissue, to keep moist for oligo application.

100 μM of HPRT-End (as in Examples 12 and 13) was diluted 1 in 20 into TE buffer to give a final concentration of 5 μM (10 μl in 200 μl TE).

A germicidal tube (UV lamp) housing was prepared. The germicidal tube was switched on 10 minutes before use to warm up. 100 μl of the HPRT-End oligo was applied to each membrane to cover the entire membrane with probe. The membranes were placed on damp tissue, then moved into the germicidal tube housing, and the probes allowed to crosslink for 2 minutes. The germicidal tube was switched off and the membranes removed from the housing and placed in a 50 ml Falcon tube with the radiated face exposed. 25 ml of 5×SSPE/0.5% SDS was added, and the membranes washed in a rotating incubator at 55° C. for 30 minutes. The 5×SSPE/0.5% SDS was then discarded.

HPRT IVT RNA for hybridisation was prepared as in Example 12, except that in these experiments the IVT was biotinylated by incorporating biotinylated nucleotides during transcription. The biotinylated IVT was used to prepare the following concentrations of RNA using RNase free water: 0.7 ug/ul, 0.6 ug/ul, 0.5 ug/ul, 0.4 ug/ul, 0.3 ug/ul, 0.2 ug/ul, 0.1 ug/ul, and 0.05 ug/ul.

10 mls of hybridisation buffer (reference 22) was prepared. The membrane was placed in a 50 ml Falcon tube and 10 ml hybridisation buffer added. The membrane was incubated at 45° C. for 30 mins or until required. The membranes were placed in a Petri dish on top of tissue which had been soaked in the warm hybridisation buffer. 3×0.5 μl of each concentration were spotted onto the membrane in columns (see FIG. 26).

The lid was placed on the Petri dish, and the dish placed in an incubator at 45° C. for 30 minutes. The membranes were removed and placed in a Falcon tube. 50 ml of a wash buffer (reference 22) was added and the Falcon tube placed on a rolling mixer and washed for 5 minutes. The membranes were transferred into 50 mls of a second wash buffer (reference 22) and washed for a further 5 minutes on the rolling mixer. The membranes were placed in a 50 ml Falcon tube, face up. 50 ml of PBS/0.1% Tween was added. The Falcon tube was placed on the rolling mixer and allowed to wash for 15 minutes. The PBS/0.1% Tween was changed for fresh PBS/0.1% Tween, and the membranes washed for a further 15 minutes.

Chemiluminescence detection was performed essentially as described for Example 12, except that 1 ml of the mixed ECL Advance detection solution was applied to each of the wet nylon membranes.

### Results & Conclusions

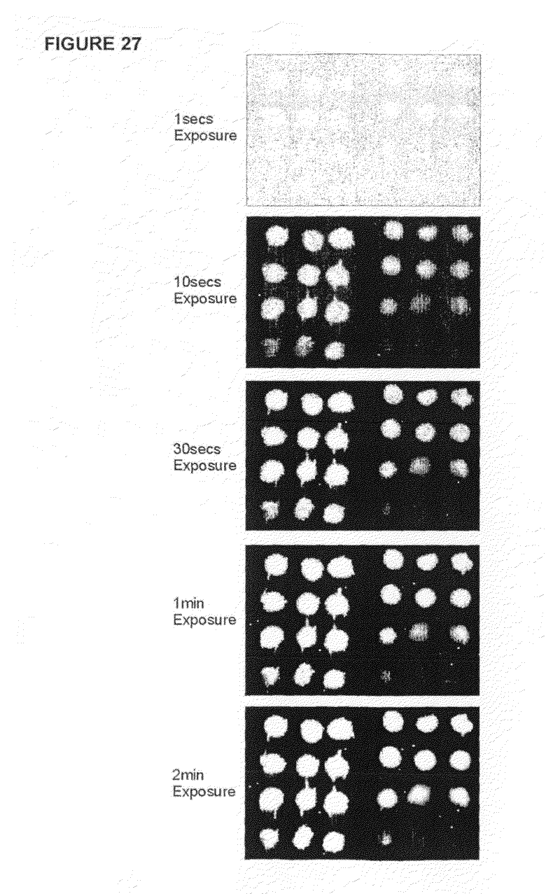

All of the concentrations of biotinylated IVT tested were detectable by chemiluminescence using HRP as substrate (see FIG. 27). These experiments provide yet further confirmation that target analytes hybridised to analytical components immobilised on a permeable support may be detected by chemiluminescence methods. These experiments allowed the limit of chemiluminescence detection to be calculated. It is expected that the limit of chemiluminescence detection could be enhanced by use of an optimised system. In summary, these experiments show that highly expressed genes can be detected by chemiluminescence even using sub-optimal detection systems. Alternative detection methods with high external quantum yield such as proximity detection on back illuminated CCD cameras should provide an even lower limit of detection, allowing even genes expressed at low levels to be detected by chemiluminescence.

It will be understood that the invention has been described by way of example only and modification of detail may be made without departing from the spirit and scope of the invention.

### REFERENCES

### The Full Contents of which are Incorporated Herein by Reference

[1] Harrington et al., Curr. Opin. Microbiol., 2000, 3 (3):285-91.
[2] WO93/22480.
[3] WO03/020415.
[4] PCT/GB2004/004390
[5] U.S. Pat. No. 6,156,576.
[6] Sims et al. (1998) Anal Chem 70:4570-7.
[7] Prinz et al. (2002) Lab Chip 2:207-12.
[8] Leffhalm et al. (2005) AKB 200.15 Di 17:00 Poster TU C. Berlin 2005, “Physik seit Einstein”, Deutsche Physikalische Gesellschaft.
[9] Przekwas et al. (2001) pages 214-217 of Modeling and Simulation of Microsystems. ISBN 0-9708275-0-4.
[10] WO2004/033629.
[11] Nie et al. (1994) Science 266:1018-21.
[12] Schmidt et al. (1996) Proc. Natl. Acad. Sci. USA 93:2926-9.
[13] Hesse et al. (2004) Anal Chem 76:5960-4.
[14] WO00/25113. See also US-2002/0030811.
[15] Lizardi et al. (1998) Nature Genetics 19, 225-232.
[16] Demidov (2005) Encyclopaedia of Diagnostic Genomics and Proteomics 1175.
[17] Dean et al. (2002) PNAS 99 (8):5261-5266.
[18] Lovmar & Syvanen (2006) Human Mutation 27 (7):603-614.
[19] Akhavan-Tafti et al. (1998) Clinical Chemistry 44 (9):2065.
[20] Rajeevan et al. (1999) J. Histochemistry & Cytochemistry 47 (3):337-342.
[21] Yin et al. (2005) Anal Chem 77:527-33.
[22] Grainger D. C et al (2005) PNAS Vol. 102 No. 49 17693-17698.

1. A process for analysing a plurality of different samples, comprising the steps of:
a) applying the samples to a support, to which an analytical component is immobilised; and
b) allowing the samples to interact with the analytical component, thus permitting analysis of the samples,
wherein the individual samples are applied in step a) to different areas of the support to produce a spatial arrangement of samples on the support, and
wherein the spatial arrangement is maintained in step b), thus permitting the results of the analysis to be matched to individual samples.

2. A process according to claim 1, further comprising matching the results of the analysis to individual samples.

3. A process for analysing a plurality of different individual cells, comprising the steps of:
a) applying material derived from individual cells to a support, to which a specific binding reagent is immobilised; and
b) allowing the material to interact with the specific binding reagent, thus permitting analysis of the material,
wherein the material derived from different individual cells is applied in step a) to different areas of the support to produce a spatial arrangement of material on the support, and wherein the spatial arrangement is maintained in step b), thus permitting the results of the analysis to be matched to individual cells.

4. A process according to claim 3, further comprising matching the results of the analysis to individual cells.

5. A process according to claim 3, wherein step a) comprises:
(i) applying cells to the support; then
(ii) releasing material from the cells.

6. A process according to claim 3, wherein step a) comprises:
(i) releasing material from the cells; then
(ii) applying the released material to the support.

7. A process according to claim 6, wherein step (i) comprises releasing material from the cells onto different areas of a substrate to produce a spatial arrangement of material on the substrate, and step (ii) comprises transferring target analytes from the substrate to the support, whilst maintaining the spatial arrangement generated in step (i).

8. A process according to claim 7, wherein the substrate is impermeable to cells or cell components but permeable to target analytes and transfer reagents.

9. A device for analysing a plurality of different individual cells, comprising a support, to which a specific binding reagent is immobilised, and on which support material derived from a plurality of different individual cells is located in a spatial arrangement that permits the results of analysis using the analytical component to be matched to individual cells.

10. A device for analysing a plurality of different individual cells, comprising:
(i) a support, to which a specific binding reagent is immobilised; and
(ii) a transfer substrate permeable to lysis reagents and target analytes but impermeable to cells or cell components,
wherein the transfer substrate is positioned against or in close proximity to the support.

11. A kit for analysing a plurality of different individual cells, comprising:
(i) a support, to which a specific binding reagent is immobilised; and
(ii) a material applicator, for applying material derived from a plurality of different individual cells to the support in a spatial arrangement that permits the results of analysis using the analytical component to be matched to individual cells.

12. The kit of claim 11, wherein the material applicator is an applicator for applying the cells to the support and then releasing material from the cells.

13. The kit of claim 11, wherein the material applicator is an applicator for releasing material from the cells and then applying the released material to the support.

14. The kit of claim 13, wherein the material applicator comprises a substrate permeable to lysis reagents and target analytes but impermeable to cells or cell components.

15. A process for analysing a plurality of different samples, comprising the steps of:
a) applying the samples to different areas of a transfer substrate to produce a spatial arrangement of samples on the transfer substrate; then
b) transferring target analytes from the transfer substrate to a support, to which a specific binding reagent is immobilised; and
c) allowing the target analytes to interact with the specific binding reagent, thus permitting analysis of the samples,
wherein the spatial arrangement of the target analytes is maintained in steps b) and c), thus permitting the results of the analysis to be matched to individual samples.

16. A process according to claim 15, further comprising matching the results of the analysis to individual samples.

17. A process according to claim 15, wherein the transfer substrate is permeable to target analytes and transfer reagents.

18. A process according to claim 17, wherein transfer reagents are applied to the transfer substrate in step b), thus permitting transfer of target analytes from the transfer substrate to the support.

19. A process according to any of claims 15 to 18, wherein the transfer substrate is positioned against or in close proximity to the support in step b), to facilitate transfer of target analytes from the transfer substrate to the support.

20. A device for analysing a plurality of different samples, comprising:
(i) a support, to which a specific binding reagent is immobilised; and
(ii) a transfer substrate permeable to target analytes and transfer reagents, positioned against or in close proximity to the support.

21. A device according to claim 20, wherein a plurality of different samples are located on the support in a spatial arrangement that permits the results of analysis using the analytical component to be matched to individual samples.

22. A kit for analysing a plurality of different samples, comprising:
(i) a support, to which a specific binding reagent is immobilised;
(ii) a transfer substrate permeable to target analytes and transfer reagents;
(iii) a material transferor for transferring target analytes from the substrate to the support, which permits a spatial arrangement of samples on the substrate to be maintained when target analytes are transferred to the support.

23. The kit of claim 22, wherein the material transferor is a transfer reagent.

24. A process, device or kit according to any preceding claim, wherein the support is impermeable to the reagents that are applied to the device during use of the device.

25. A process, device or kit according to any of claims 1-23, wherein the support is permeable to the reagents that are applied to the device during use of the device.

26. A process, device or kit according to any preceding claim, wherein the specific binding reagent is a nucleic acid.

27. A process, device or kit according to any of claims 1 to 25, wherein the specific binding reagent is an antibody or antibody fragment.

28. A process, device or kit according to any of claims 1 to 25, wherein the specific binding reagent is an aptamer.

29. A process, device or kit according to any of claims 1 to 25, wherein the specific binding reagent is a small molecule.

30. A process, device or kit according to claim 29, wherein the small molecule is an organic molecule with a molecular weight of less than 2000 Daltons.

31. A process, device or kit according to claim 29, wherein the small molecule is a peptide or peptide analog comprising at least 5 amino acid residues.

32. A process, device or kit according to any of claims 1 to 25, wherein different specific binding reagents are immobilised in patches on the support.

33. A process, device or kit according to claim 32, wherein a single cell is applied to a patch of specific binding reagent.

34. A process, device or kit according to claim 32, wherein each patch is sized to permit parallel analysis of at least two samples.

35. A process, device or kit according to claim 34, wherein at least two cells are applied to a patch of specific binding reagent.

36. A process, device or kit according to any of claims 1 to 25, wherein each individual sample comprises a single cell.
